(19) United States
(12) Patent Application Publication
(10) Pub. No.: US 2018/0170849 A1
(43) Pub. Date: Jun. 21, 2018

(54) PHOTOLABILE PRO-FRAGRANCES

(71) Applicant: Henkel AG & Co. KGaA, Düsseldorf (DE)

(72) Inventors: Thomas GERKE, Düsseldorf (DE); Christian KROPF, Hilden (DE); Ursula HUCHEL, Cologne (DE); Axel GRIESBECK, Cologne (DE); Björn PORSCHEN, Cologne (DE)

(21) Appl. No.: 15/737,337

(22) PCT Filed: Jun. 2, 2016
(86) PCT No.: PCT/EP2016/062501

§ 371 Date: Dec. 18, 2017

(30) Foreign Application Priority Data
Jun. 17, 2015 (DE) 10 2015 211 137.7

(51) Int. Cl. C07C 45/29; C07C 67/343; C07C 29/147; C07C 45/62; C07C 29/40; C07C 49/798; C11B 9/00

(52) U.S. Cl. CPC C07C 45/292; C07C 67/343; C07C 29/147; C07C 45/298; C07C 2601/16; C07C 29/40; C07C 49/798; C11B 9/0061; C07C 2601/14; C07C 45/62

(57) ABSTRACT

A method for producing photo-cleavable fragrance pre-cursors is described, which includes potential stereoselective method steps, agents containing the fragrance pre-cursors and the use of the fragrance pre-cursors for prolonging the scent impression in the agent and on surfaces treated with said agent.

### CROSS-REFERENCE TO RELATED APPLICATIONS

The present application is a national stage entry according to 35 U.S.C. § 371 of PCT Application No. PCT/EP2016/062501 filed on Jun. 2, 2016, which claims priority to German Patent Application No. 102015211137.7, filed on Jun. 17, 2015; both of which are herein incorporated by reference in their entirety.

### TECHNICAL FIELD

The subject matter herein generally relates to a method for preparing pro-fragrances, selected pro-fragrances themselves and agents use of such pro-fragrances, and more specifically to the use of pro-fragrances for prolonging a scent impression.

### BACKGROUND

Washing or cleaning agents, cosmetic agents, adhesives or printing inks contain mostly fragrances which endow the agents with a pleasant odor. In so doing, the fragrances mask not only a possible odor of the other ingredients, but also allow a pleasant impression of an odor to form for the consumer.

In particular in the field of washing agents and cleaning agents, fragrances are important components of the composition, as the laundry is intended to have a pleasant and possibly also a fresh smell, both when wet and also when dry.

In principle, when using fragrances in washing agents or cleaning agents, cosmetic agents, adhesives or printing inks, one is faced with the problem that the fragrances are more or less slightly volatile compounds, but the aim is to achieve a long-lasting scent impression. In particular in those fragrances which represent the fresh and light notes of the perfume, and evaporate particularly quickly as a result of their high vapor pressure, the desired longevity of the scent impression is hard to achieve.

The use of so-called photoactivatable substances as pro-fragrances represents a possibility for delayed release of fragrances. The incidence of sunlight or other electromagnetic radiation of a specific wavelength causes a covalent bond in the pro-fragrance molecule to break, as a result of which a fragrance is released.

In this context, WO 2011/101179 A1 discloses special ketones as photoactivatable substances which, in the presence of sunlight, release a fragrance with at least one cyclic double bond in a photochemical fragmenting.

In so doing, proceeding from the fragrance, the named pro-fragrances are prepared in a two-stage synthesis via a hydroboration of the at least one cyclic double bond and a subsequent substitution reaction.

A disadvantage of the synthesis method described in WO 2011/101179 A1 is that fragrances which, in addition to the at least one cyclic double bond, additionally possess one or more semicyclic and/or exocyclic double bonds, can be converted to the desired pro-fragrances, non-selectively or not at all as, during the hydroboration of the fragrance, the sterically least impeded double bond reacts, regioselectively, first. A disadvantage of these undesired constitution isomers is that they are cleaved only insufficiently or not at all when illuminated with light, and do not release the stored fragrance again.

In view of this disadvantage, WO 2014/191256 A1 provides a synthesis method for such pro-fragrances which does not relate to the regioselective hydroboration of the underlying fragrances. Instead, the pro-fragrances are produced proceeding from a cyclic ketone corresponding to the latent fragrance structure unit and a phosphonium ylide generated in situ in a three-stage synthesis, wherein a regioselective reduction with sodium borohydride, carried out in the presence of Pd metal, supplies the desired pro-fragrance after generating an α,β-unsaturated ketone.

In spite of the advantage of preventing a regioselective hydroboration, the synthesis route described in WO 2014/191256 A1 has specific disadvantages. Inter alia, the route requires the commercial availability of benzonitrile coupling partners (or the respective synthesis thereof) and, for commercial or other reasons, there is often the need to produce the starting phosphonate, in particular if the synthesis is carried out on a large scale. This in turn requires the use of toxic, potentially carcinogenic alkylating agents and unconventional reaction technologies, such as the microwave technology applied in WO 2014/191256 A1. The use of such technologies can in turn prove problematic if their use is required on a large scale. Additionally, the synthesis path described in WO 2014/191256 A1 requires the use of expensive heavy metals in the last method step.

When providing fragrances, it is of utmost importance that precisely the desired molecules are provided. Small differences at the molecular level, such as for example different double-bond regiochemistry or stereocenter configurations, can lead to fragrance properties which differ greatly from the intended or desired fragrance profile. When using pro-fragrances it is therefore optimal if the eventual cleavage of the fragrance molecule opens exclusively in the release of the desired fragrance. In this context, the release of undesired fragrances is minimized if the pro-fragrance itself represents only one single absolute compound, i.e. in cases in which a pro-fragrance contains one or more stereocenters in order to produce a unified cleavage, and thus fragrance, profile may be used as a single isomer (diastereomer, enantiomer). In light of the strict requirements in respect of the regulatory process, and within the scope of the characterizing mandatory identifications, it is advantageous, in particular in the field of washing agents and cleaning agents, if product components can be produced and used as individual compounds. In this sense, stereoselective syntheses represent the optimal approach because they lead to diastereomerically and/or enantiomerically enriched and possibly even to diastereomer- and/or enantiomer-pure products, and thus reduce, or even indeed completely remove, the need for complex and/or expensive purification methods. As a result of the simplified purification and corresponding analysis methods, also the introduction of undesired by-products in consumer products is prevented or at least clearly reduced.

### SUMMARY

According to a non-limiting embodiment, a method for producing a ketone of the general formula (I) is disclosed.

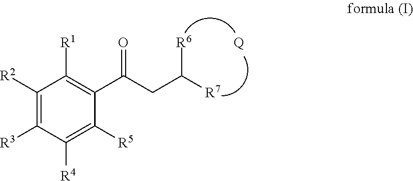

R¹ to R⁵, in each case independently of one another, may stand for hydrogen, halogen, NO₂—, —OH, —NR′R″R′″, or for linear, branched, or cyclic, saturated or unsaturated radicals with up to 15 carbon atoms, wherein R′, R″, and R′″, in each case independently of one another, may be selected from hydrogen and up to 15 carbon atoms, where the carbon atoms are linear, branched, cyclic, saturated, unsaturated alkyl radicals, or combinations thereof. The carbon atoms of the ring of formula (I) linked to R¹ and R² or R² and R³ may be a common component of an annealed five-, six- or seven-membered cycloalkyl, aryl, heteroaliphatic or heteroaryl ring, where the radicals R¹ and R² or R² and R³, in each case independently of one another, are completely or partly an integral component of the annealed ring. One or more hydrogen atoms, methyl groups, methylene groups, methine groups, quaternary carbon atoms, or combinations thereof of the radicals R¹ to R⁵ can, in each case independently of one another, may be substituted by heteroatoms.
R⁶ to IC, in each case independently of one another, may be a secondary, tertiary, or quaternary carbon atom. Q may be an R⁶ to IC-bridging substituted or unsubstituted group with up to 11 carbon atoms. The method may include:
a) obtaining a ketone of the general formula (II)

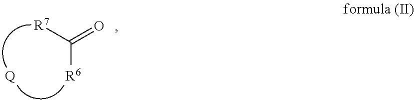

wherein the radicals R⁶, IC and Q are identical to the general formula (I), with a phosphonate of the general formula (III)

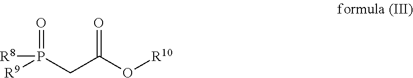

wherein R⁸ and R⁹, in each case independently of one another, stand for an alkoxy group with up to 15 carbon atoms, and R¹⁰ stands for an alkyl group with up to 10 carbon atoms, is converted to an alpha,beta-unsaturated ester of the general formula (IV)

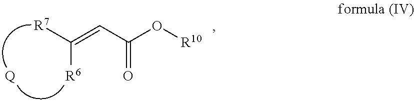

wherein one or more hydrogen atoms, methyl groups, methylene groups, methine groups, or quaternary carbon atoms of the radicals R⁸ and R⁹ can, in each case independently of one another, be substituted by heteroatoms,
b) reducing the alpha,beta-unsaturated ester of the general formula (IV) obtained in step a) to an alcohol of the general formula (V)

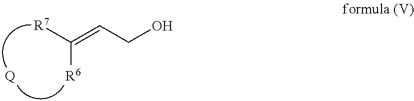

c) oxidizing the allyl alcohol of the general formula (V) obtained in step b) to an alpha,beta-unsaturated aldehyde of the general formula (VI)

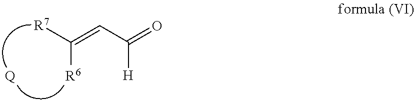

d) reducing the alpha,beta-unsaturated aldehyde of the general formula (VI) obtained in step c) to an aldehyde of the general formula (VII)

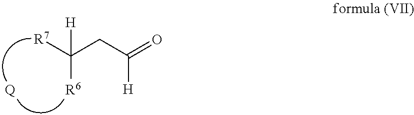

e) converting the aldehyde of the general formula (VII) obtained in step d) to an alcohol of the general formula (VIII)

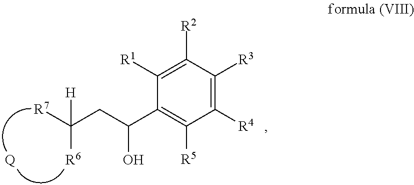

wherein the radicals R¹ to R⁵ are identical to the general formula (I) and
f) oxidizing the alcohol of the general formula (VIII) obtained in step e) to the ketone of the general formula (I).

According to yet another non-limiting embodiment, a ketone is obtained having the general formula of (X) or (XI).

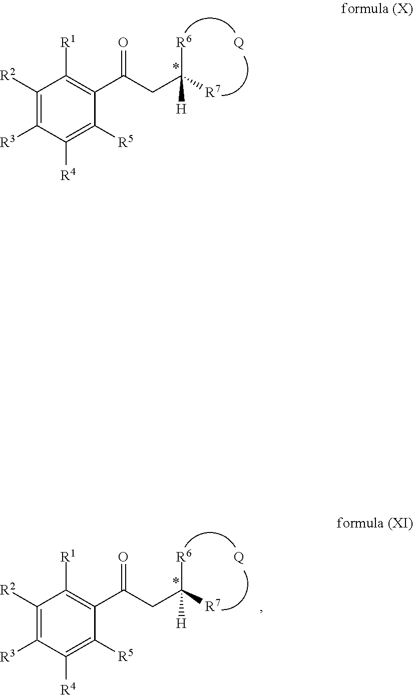

R¹, R², R³, R⁴, R⁵, R⁶, R⁷ and Q may be as defined above with respect to the method, * indicates a stereocenter, and the proportion of the represented epimer in relation to the corresponding other epimer is greater than 50%.

According to yet another non-limiting embodiment, an agent comprising a ketone is obtained having the general formula of (X) or (XI). The agent may be a washing agent, a cleaning agent, a cosmetic agent, an adhesive, a printing ink, or combinations thereof.

### DETAILED DESCRIPTION

A simple, efficient, convergent method for selective preparation of a pro-fragrance is described, which on the one hand guarantees an effective, specific release of the stored fragrance, and which on the other hand can store fragrances which, in addition to at least one cyclic double bond, can additionally have semicyclic and/or exocyclic double bonds. More specifically, a method for selective preparation of a pro-fragrance may overcome one or more of the above-named disadvantages of the methods described in the state of the art, such as for example (e.g.) the use of toxic and/or expensive chemicals, heavy metals and so forth. In addition, a method for producing a pro-fragrance may be designed to be non-stereoselective or also stereoselective, e.g. by using corresponding non-chiral (achiral) or chiral catalysts/reagents. Yet another object is the provision of a stereoselective method for preparing the above-named pro-fragrances.

This object has surprisingly been achieved by a method for producing a ketone of the general formula (I)

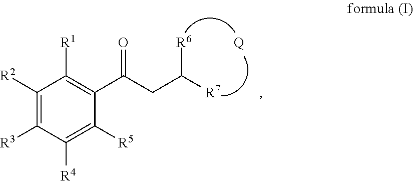

wherein R¹ to R⁵, in each case independently of one another, stand for hydrogen, halogen, —NO₂—, —OH, —NR′R″R′″, or for linear, branched, or cyclic, saturated or unsaturated radicals with up to 15 carbon atoms, wherein R′,R″, and R′″, in each case independently of one another, are selected from hydrogen and linear, branched, or cyclic, saturated or unsaturated alkyl radicals with up to 15 carbon atoms, and wherein

the carbon atoms of the ring of formula (I) linked to R¹ and R² or R² and R³ can be a common component of an annealed five-, six- or seven-membered cycloalkyl, aryl, heteroaliphatic or heteroaryl ring, wherein the radicals R¹ and R² or R² and R³, in each case independently of one another, are completely or partly an integral component of the annealed ring; and wherein one or more hydrogen atoms, methyl groups, methylene groups, methine groups or quaternary carbon atoms of the radicals R¹ to R⁵ can, in each case independently of one another, be substituted by heteroatoms; and

wherein R⁶ to IC, in each case independently of one another, stand for a secondary, tertiary or quaternary carbon atom; and wherein

Q stands for a R⁶ to R⁷-bridging substituted or unsubstituted group with 1 or 2 to 11 carbon atoms;

characterized in that

a) a ketone of the general formula (II)

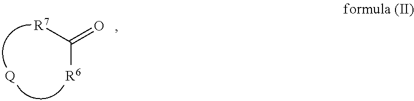

wherein the radicals R⁶, R⁷ and Q are identical to the general formula (I) and, with a phosphonate of the general formula (III)

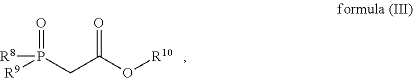

wherein R⁸ and R⁹, in each case independently of one another, stand for an alkoxy group with 1 or 2 to 15 carbon atoms and R¹⁰ stands for an alkyl group with 1 or 2 to 10 carbon atoms, is converted to an alpha,beta-unsaturated ester of the general formula (IV)

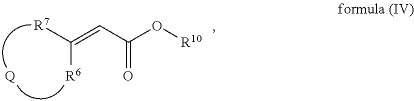

wherein one or more hydrogen atoms, methyl groups, methylene groups, methine groups or quaternary carbon atoms of the radicals R⁸ to R⁹ can, in each case independently of one another, be substituted by heteroatoms,

b) the alpha,beta-unsaturated ester of the general formula (IV) obtained in step a) is reduced to an alcohol of the general formula (V)

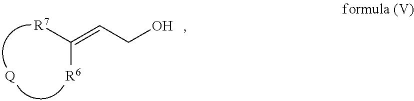

c) the allyl alcohol of the general formula (V) obtained in step b) is oxidized to an alpha,beta-unsaturated aldehyde of the general formula (VI)

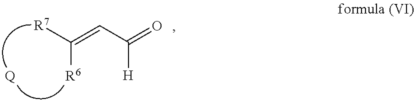

d) the alpha,beta-unsaturated aldehyde of the general formula (VI) obtained in step c) is reduced to an aldehyde of the general formula (VII)

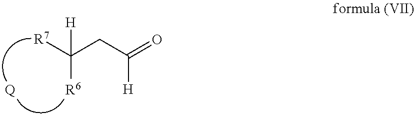

e) the aldehyde of the general formula (VII) obtained in step d) is converted to an alcohol of the general formula (VIII)

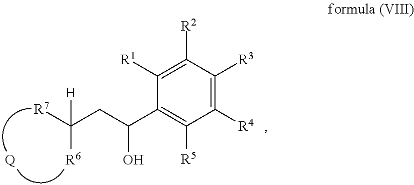

wherein the radicals R¹ to R⁵ are identical to the general formula (I) and f) the alcohol of the general formula (VIII) obtained in step e) is oxidized to the ketone of the general formula (I).

The term secondary carbon atom is understood to mean a carbon atom which is covalently bonded to two further carbon atoms. The term tertiary carbon atom or quaternary C atom is understood to mean a carbon atom which is covalently bonded to three or four further carbon atoms, where a tertiary carbon atom or a quaternary carbon atom can also be a carbon atom which is covalently bonded to only two (tertiary) or three (quaternary) further carbon atoms and forms a double bond with one of the two or three carbon atoms.

If one or more of the radicals R¹ to R⁵ stands for linear, branched, or cyclic, saturated or unsaturated radicals with up to 15 carbon atoms, independently of one another the radicals stand for alkyl, alkenyl, cycloalkyl, acyl, aryl or heteroaryl groups. An acyl radical is a radical which is bonded to the benzene ring of the structure of formula (I) by a carbonyl group. Aryl groups and heteroaryl groups include inter alia monocyclic, bicyclic and tricyclic groups. Aryl groups can inter alia be benzene groups and naphthalene groups which are potentially further substituted. Heteroaryl groups can inter alia be N-containing heteroaryl rings which contain up to 2 further heteroatoms selected from the list of N, O and S. For example, such heteroaryl rings can be pyrrole, pyrazole, imidazole, oxazole, isoxazole, thiazole, isothiazole, triazole, oxadiazole, thiadiazole, pyridine, pyridazine, pyrimidine, pyrazine, triazine or indole rings. The heteroaryl groups can be substituted or unsubstituted.

The carbon atoms of the ring of formula (I) linked to R¹ and R² or to R² and R³ can be a common component of an annealed five-, six- or seven-membered ring, wherein the radicals R¹ and R², in each case independently of one another, are completely or partly an integral component of the annealed ring. Annealed means, in this context, that two carbon rings share a carbon-carbon bond, specifically those carbon atoms of the ring of formula (I) linked to R¹ and R², wherein this bond can be a single or double bond. If the radicals R¹ and R² or R² and R³ are only a partially integral component of the annealed ring, the non-integral components of the ring of the radicals R¹ and R² can be present, for example in the form of a side chain of the annealed ring.

One or more hydrogen atoms, methyl groups, methylene groups, methine groups or quaternary carbon atoms of the radicals R¹ to R⁵ of the pro-fragrance (photocage) can, in each case independently of one another, be substituted by heteroatoms. Heteroatoms may be or include nitrogen, oxygen, sulfur, silicon, selenium, phosphorus, fluorine, chlorine, bromine, iodine, or combinations thereof. One or more methyl groups can be substituted by a heteroatom selected from the group comprising nitrogen, oxygen, sulfur, silicon, selenium, phosphorus, fluorine, chlorine, bromine or iodine, one or more methylene groups can be substituted by a heteroatom selected from the group comprising nitrogen, oxygen, sulfur, selenium, phosphorus, or silicon, one or more methine groups can be substituted by a heteroatom selected from the group comprising nitrogen, phosphorus or silicon and one or more quaternary carbon atoms can be substituted by silicon. If one or more methyl groups, methylene groups, methine groups or quaternary carbon atoms of the radicals R¹ to R⁵ are substituted by heteroatoms, this means that the corresponding group is exchanged for a heteroatom. If free valences arise as a result of the substitution of a methyl group, a methylene group or a methine group, these are in principle saturated with hydrogen. A terminal methyl group in addition to a methylene group can thus for example be exchanged for a hydroxy group or a sulfanyl group, with the result that a methylene hydroxy group or a methylene thiol group is obtained. By analogy with this, an isopropyl group which is a radical with two methyl groups and one methine group or a derivative of the isopropyl group which is a radical with one methyl group, one methylene group and one quaternary carbon atom, can for example have the following substitution pattern:

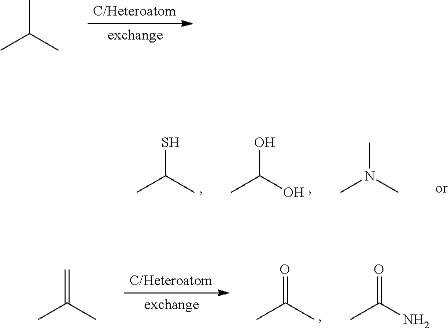

Methyl groups, methylene groups, methine groups or quaternary carbon atoms of the radicals R¹ to R⁵ can in principle be substituted by heteroatoms, but with the exception of di- or polysulfides, no two directly adjacent groups are substituted simultaneously by heteroatoms.

It has surprisingly been able to be found that, due to the method, pro-fragrances of the general formula (I) can be obtained simply, in which pro-fragrances fragrances with at least one cyclic double bond are stored and which effectively release again the stored fragrance with at least one cyclic double bond by radiation with light.

The term “fragrance” is not the ketone of the general formula (II) for synthesizing the pro-fragrance of the general formula (I), but the compound which is released from the pro-fragrance when illuminated. This distinguishes the present method from the method described in WO 2011/101179 A1, where the fragrance to be released is used as a starting compound.

The term “endocyclic double bond”/“cyclic double bond” is understood to mean a double bond in which both of the two atoms connected by the double bond represent ring atoms. The term “exocyclic double bond” is understood to mean a double bond in which none of the two atoms connected by the double bond represents a ring atom. The term “semicyclic double bond” is understood to mean a double bond in which one of the two atoms connected by the double bond represents a ring atom and the other lies outside of the ring. For example:

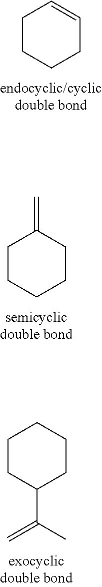

In a non-limiting embodiment, the ketone of the general formula (II) has at least one semicyclic or exocyclic double bond.

Fragrances with at least one cyclic double bond and at least one semicyclic or exocyclic double bond represent a particular requirement. Methods known from the state of the art include conventionally a hydroboration step which takes place at the sterically least hindered double bond. The selective storage of corresponding fragrances in pro-fragrances of the general formula (I) is therefore problematic. In contrast, regardless of the number of semicyclic or exocyclic double bonds of the released fragrance, the method makes possible the selective representation of the desired pro-fragrance of the general formula (I).

If R⁶ and/or IC stand for a tertiary or quaternary carbon atom, the exocyclic non-hydrogen atoms bonded to R⁶ and/or R⁷ are, independently of one another, carbon atoms which are part of a saturated or unsaturated, straight-chained or branched hydrocarbon group, such as an alkyl group with up to 4 carbon atoms. The exocyclic non-hydrogen atoms bonded to R⁶ and/or R⁷ can be part of a methyl group. The exocyclic non-hydrogen atoms bonded to R⁶ and/or R⁷ can be part of an ethyl group. The exocyclic non-hydrogen atoms bonded to R⁶ and/or R⁷ can be part of an n-propyl group. The exocyclic non-hydrogen atoms bonded to R⁶ and/or R⁷ can be part of an i-propyl group. The exocyclic non-hydrogen atoms bonded to R⁶ and/or R⁷ can be part of an n-butyl group. The exocyclic non-hydrogen atoms bonded to R⁶ and/or R⁷ can be part of an i-butyl group. The exocyclic non-hydrogen atoms bonded to R⁶ and/or R⁷ can be part of an s-butyl group. The exocyclic non-hydrogen atoms bonded to R⁶ and/or R⁷ can be part of a t-butyl group.

If Q is substituted, Q is substituted by one or more groups which, independently of one another, are bonded to Q by C, O, N or S atoms contained in the group, bonded by C atoms contained in the group. If Q is bonded directly to a C atom contained in the group, the group may be a straight-chained or branched, saturated or unsaturated hydrocarbon group with up to 6 carbon atoms. If Q is substituted, Q can be substituted by an isopropenyl group.

If R⁶ and/or IC and/or Q are substituted, stereocenters can result from the substitutions. Such stereocenters can have the (R) and (S) configurations, as well as mixtures of the (R) and the (S) configuration.

In a non-limiting embodiment, the bridging part —R⁷-Q-R⁶— of the ketone of the general formula (II) is a hydrocarbon.

The corresponding fragrances to the above-described ketone of the general formula (II), in which the carbonyl group of the ketone of the general formula (II) is a methylene or methine group, are characterized by their high vapor pressure and, because of their often-low degree of functionalization, can bind, chemically, to conventional carrier media only with difficulty. Now it is possible, using the method, to provide pro-fragrances which release these fragrances in targeted manner over a longer period of time.

In a non-limiting embodiment, the ketone of the general formula (II) is dihydrocarvone (5-isopropenyl-2-methylcyclohexanone). Dihydrocarvone possesses two stereocenters. The ketone of the general formula (II) may be or include, but is not limited to, (2 S,5 S)-5-isopropenyl-2-methylcyclohexanone ((−)-dihydrocarvone), (2 S,5R)-5-isopropenyl-2-methylcyclohexanone ((+)-isodihydrocarvone), (2R,5R)-5-isopropenyl-2-methylcyclohexanone ((+)-dihydrocarvone, (2R,5S)-5-isopropenyl-2-methylcyclohexanone ((−)-isodihydrocarvone), or mixtures thereof. In a non-limiting embodiment, the ketone of the general formula (II) is (2S,5R)-5-isopropenyl-2-methylcyclohexanone ((+)-isodihydrocarvone), or (2R,5R)-5-isopropenyl-2-methylcyclohexanone ((+)-dihydrocarvone), or mixtures thereof.

According to the method, a pro-fragrance of the general formula (I) is obtained from (+)-dihydrocarvone and/or (+)-isodihydrocarvone, which pro-fragrance, when illuminated with light, releases limonene which is one of the most important fragrances in the field of washing agents and cleaning agents. The odor of limonene is frequently associated with freshness, which for the consumer is synonymous with cleanliness and purity.

In order to achieve an optimal conversion of carbonyl compounds to olefins by means of a Horner-Wadsworth-Emmons (HWE) reaction or similar phosphonate anion-mediated olefination, it is advantageous to use a phosphonate where the reaction conditions have a balanced ratio of solubility, stability and reactivity.

In a further non-limiting embodiment, the radicals R⁸ and R⁹ of the phosphonate of the general formula (III) are, in each case independently of one another, methoxy, ethoxy, n-propoxy, i-propoxy, n-butoxy, i-butoxy, s-butoxy, or t-butoxy radicals.

In a further non-limiting embodiment, the radical R¹⁰ of the phosphonate of the general formula (III) may be or include, but is not limited to, a methyl, ethyl, n-propyl, i-propyl, n-butyl, sec-butyl, i-butyl or t-butyl radical. Ethoxy radicals may be of particular interest.

As mentioned above, preventing the use of expensive and potentially toxic and/or environmentally harmful reagents and metals is advantageous in industrial chemistry and in particular in the consumer product field. In a non-limiting embodiment, the reduction in method step d) therefore takes place in the presence of an organic catalyst.

As mentioned above, supplying pro-fragrances as individual compounds is of great interest, for numerous reasons. In a further non-limiting embodiment, the reduction in method step d) is therefore stereoselective.

In a further non-limiting embodiment, the reduction in method step d) takes place in the presence of a chiral catalyst, in particular of a chiral organic catalyst.

In a further non-limiting embodiment, the reduction in method step d) takes place in the presence of a chiral imidazolidinone.

In a non-limiting embodiment, in method step e) a halide according to the general formula (IX) is used,

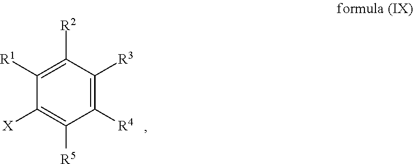

which halide is transformed into a metal organyl and then reacted with the aldehyde of the formula (VII), wherein X stands for —F, —Cl, —Br, —I and the radicals R¹ to R⁵ are as defined in claim 1 and in particular, in each case independently of one another, stand for hydrogen, methyl, isopropyl or methoxy groups, wherein methyl radicals may be used and wherein the metal is selected from the group Mg, Zn, Ce and Li.

In a further non-limiting embodiment, the ketone of the general formula (I) is obtained with an enantiomer and/or diastereomer excess and has the structure of the formula (X) or (XI), wherein R¹ to R⁷ and Q are as defined in claim 1, * indicates a stereocenter and the proportion of the represented epimer in relation to the corresponding other epimer is greater than 50%.

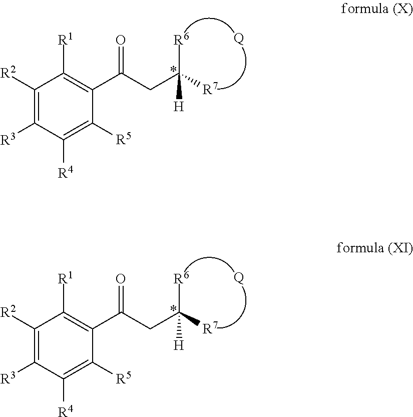

In a further non-limiting embodiment the ketone of the general formula (I) has at least one of the general formulae (XII) to (XIX), wherein R¹ to R⁵ are as defined in claim 1.

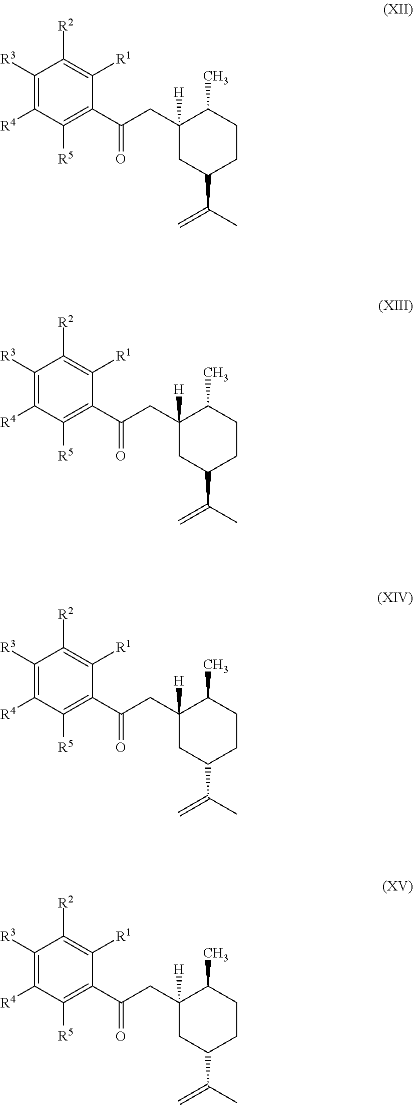

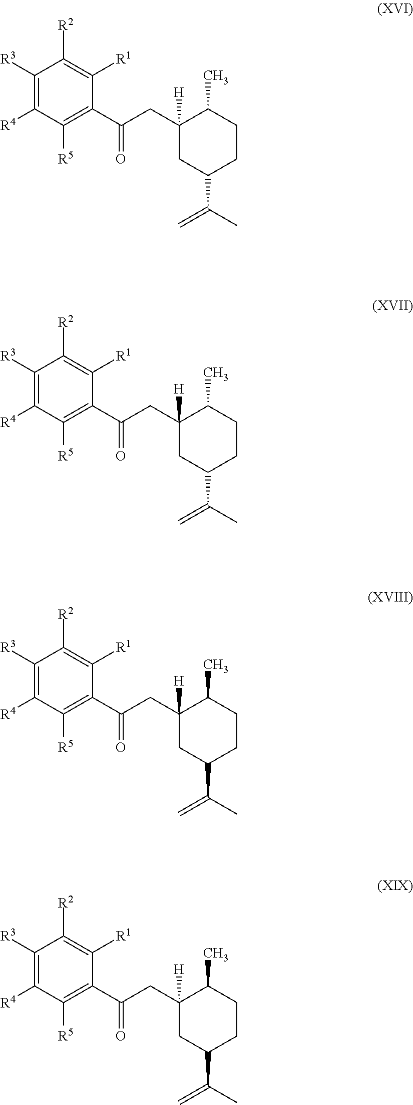

In a non-limiting embodiment the ketone has at least one of the structures (XII), (XIII), (XVIII) or (XIX), wherein (XII) and (XIII) are of particular use.

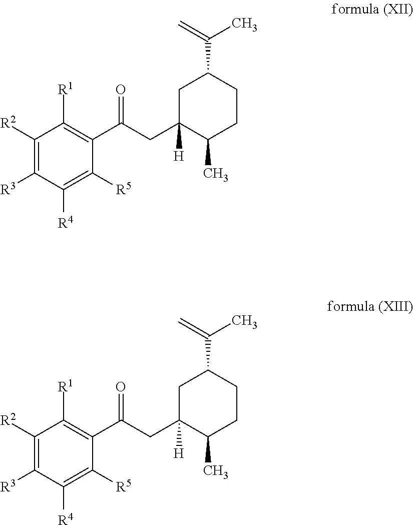

A further non-limiting embodiment is a ketone of one of the general formulae (X) or (XI),

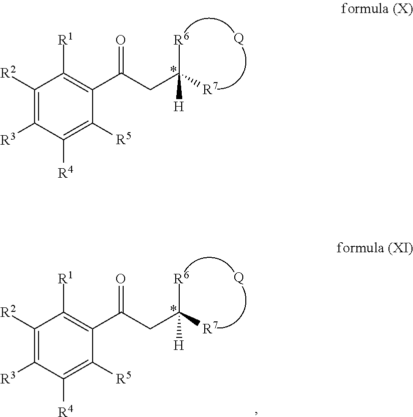

wherein R¹, R², R³, R⁴, R⁵, R⁶, R⁷ and Q are as defined above, * indicates a stereocenter, and the proportion of the represented epimer in relation to the corresponding other epimer is greater than 50%, which can be produced by the method.

The ketone of the formula (X) or (XI) may have at least one of the general formulae (XII) to (XIX), wherein R¹ to R⁵ are as defined above. Particularly, the ketone has at least one of the structures (XII), (XIII), (XVIII) or (XIX), wherein (XII) and (XIII) may be of particular use.

Ketones of the general formulae (XII), (XIII), (XVIII) and (XIX) are particularly interesting from an olfactory point of view, as the stored fragrance (limonene) is one of the most important base components of many perfume compositions. Free limonene consists exclusively of carbon and hydrogen, is slightly volatile and, in addition to its cyclic double bond, possesses an exocyclic double bond. Therefore, until now it has been challenging to incorporate limonene and/or “latent limonene” using the synthesis routes known in the state of the art, to produce pro-fragrances in same.

A further non-limiting embodiment, an agent containing a ketone of the general formula (X) or (XI) may be obtained, which can be produced by the method as described above for ketones of the general formula (I),

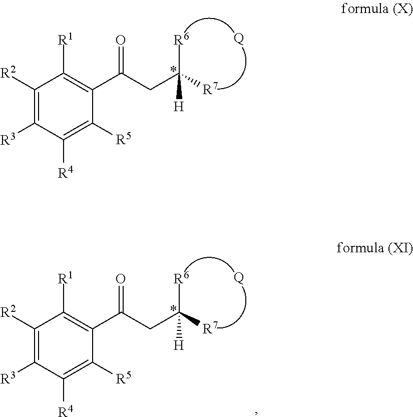

wherein R¹, R², R³, R⁴, R⁵, R⁶, R⁷ and Q are as defined above, * indicates a stereocenter, and the proportion of the represented epimer in relation to the corresponding other epimer is greater than 50%.

An agent, containing a ketone of the general formula (X) or (XI), supplies, also after a long storage period, a pleasant scent impression, as the fragrances stored in the pro-fragrances are released again initially by radiation with preferably natural sunlight or commercially available light sources. In this way, a premature evaporation of the fragrance is prevented.

In a non-limiting embodiment, the agent is a washing agent or cleaning agent, cosmetic agent, adhesive or a printing ink.

In a further non-limiting embodiment, the ketone of the general formula (X) or (XI) used in the agent is at least one ketone of one of the general formulae (XII) to (XIX), such as at least one of the general formulae (XII), (XIII), (XVIII) or (XIX), such as of the general formula (XII) or (XIII).

In particular in washing agents or cleaning agents, a fresh scent impression is expressly desired by consumers. A washing agent or cleaning agent containing at least one of the ketones of the general formulae (XII), (XIII), (XVIII) and (XIX) is therefore particularly advantageous as the ketone is a limonene store and the odor of limonene is associated with freshness by consumers.

In a further non-limiting embodiment, the agent contains a ketone of the general formula (X) or (XI) in quantities between 0.0001 and 10 wt.-%, advantageously between 0.0005 and 5 wt.-%, more advantageously between 0.001 and 3 wt.-%, in particular between 0.005 and 2 wt.-%, said wt.-% in each case relative to the whole agent.

As a ketone of the general formula (I) possesses a considerably lower vapor pressure than its corresponding fragrance, smaller quantities of the pro-fragrance can be used than of the fragrance itself in order to achieve a long-lasting scent impression, which is advantageous from an ecological and economical point of view.

A further non-limiting embodiment, is the use of a ketone of the general formulae (X) or (XI)

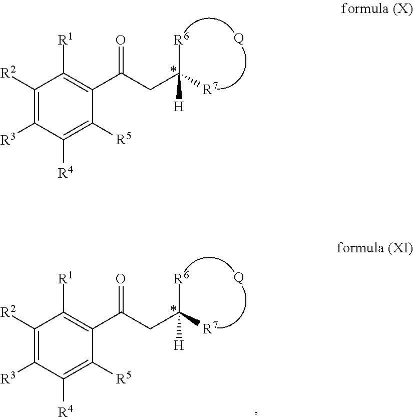

wherein R¹, R², R³, R⁴, R⁵, R⁶, R⁷, Q and * are as defined above and the proportion of the represented epimer in relation to the corresponding other epimer is greater than 50%, which can be produced by the method, in a washing agent or cleaning agent, cosmetic agent, adhesive or a printing ink for prolonging the scent impression of the washing agent or cleaning agent, cosmetic agent, adhesive or printing ink.

A further non-limiting embodiment, includes the use of a ketone of the general formulae (X) or (XI),

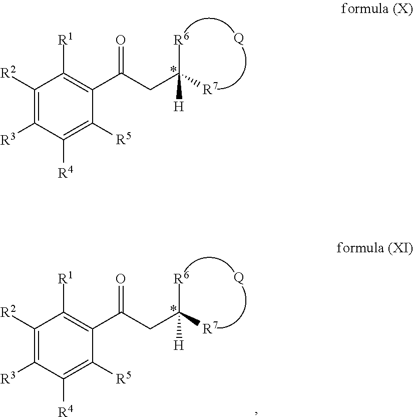

wherein R¹, R², R³, R⁴, R⁵, R⁶, R⁷, Q and * are as defined above and the proportion of the represented epimer in relation to the corresponding other epimer is greater than 50%, which can be produced by the method, for prolonging the scent impression on a surface treated with a washing agent or cleaning agent, cosmetic agent, adhesive or printing ink.

If the proportion of an epimer in relation to the corresponding other epimer is greater than 50%, the proportion of the epimer present in excess is such as at least 60%, such as at least 70%, more such as at least 80%, even more such as at least 90%, in particular at least 95%, particularly such as at least 99%. If the proportion of an epimer in relation to the corresponding other epimer is greater than 50%, alternatively, the proportion of the epimer present in excess 99.5% or 99.9% to 100%.

A ketone of the general formula (X) or (XI) may be obtained having at least one of the general formulae (XII) to (XIX), such as at least one of the general formulae (XII), (XIII), (XVIII) or (XIX), such as one of the general formula (XII) or (XIII).

The invention is intended to be described in further detail below, inter alia with the help of examples.

Producing a ketone of the general formula (I) is a multi-stage method in which firstly a ketone of the general formula (II) is converted into an alpha,beta-unsaturated ester of the general formula (IV) in the presence of a phosphonate of the general formula (III). Subsequently, the obtained alpha,beta-unsaturated ester of the general formula (IV) is selectively reduced to an alcohol of the general formula (V), which in turn is oxidized to an alpha,beta-unsaturated aldehyde of the general formula (VI). A regioselective 1,4-reduction of the alpha,beta-unsaturated aldehyde of the general formula (VI) supplies an aldehyde of the general formula (VII) and can be carried out either in a non-stereoselective or in a stereoselective manner. The conversion of the product aldehyde of the general formula (VII) to an alcohol of the general formula (VIII) is followed by the oxidation of the product alcohol (VIII) to a ketone of the general formula (I).

In a non-limiting embodiment, the conversion of the ketone of the general formula (II) with the phosphonate of the general formula (III) into an alpha,beta-unsaturated ester of the general formula (IV) is carried out in a solvent. Suitable solvents are toluene and aprotic polar solvents such as for example tetrahydrofuran (THF), diethyl ether, dichloromethane, dimethylformamide (DMF), ethylene glycol dimethyl ether (DME) or mixtures thereof. In particular THF, diethyl ether, toluene and DME are particularly non-limiting solvents for use with the method.

The reaction may be carried out in the presence of a base. There can be used as base a suitable one known to a person skilled in the art which can deprotonate the phosphonate of the general formula (III). Strong bases, such as for example alkali-metal hydrides, alkali metal bis(trimethylsilyl)amide, alkali metal alcoholates or organolithium bases, in particular sterically hindered organolithium bases such as lithium diisopropyl amide (LDA) or tert-butyl lithium, are used, wherein the base is not limited to the examples named here. Particularly preferred are alkali metal hydrides such as sodium hydride as well as LDA.

The reaction temperature may range between −100° C. and 100° C. and is correspondingly adapted to the reactivity of the individual reagents. The temperature of the reaction solution can for example initially be −78° C. and be increased, stepwise, in the course of the reaction to for example room temperature, thus 20 to 25° C. These conditions may be used with organolithium bases such as lithium diisopropyl amide (LDA) or tert-butyl lithium, and when using alkali-metal bis(trimethylsilyl)amide. Similarly, the temperature of the reaction solution can for example initially be 0° C. and be increased, stepwise, in the course of the reaction to for example room temperature, thus 20 to 25° C. Such conditions may be used with alkali metal hydrides such as sodium hydride. During the deprotonation of the phosphonate (III), the temperature of the reaction mixture can also be increased, e.g. from −78° C. to −40° C. or −20° C. or 0° C. or from 0° C. to 10° C. or room temperature, and reduced again before introducing the ketone (II).

The ketone of the general formula (II) is used, in respect of the phosphonate of the general formula (III), in a ratio of from 1:20 to 20:1, such as from 1:10 to 10:1, such as from 1:5 to 5:1, alternatively from 1:2 to 2:1.

If necessary, the purification of the formed alpha,beta-unsaturated ester of the general formula (IV) takes place by distillation, crystallization and/or chromatographic separating methods which are known to a person skilled in the art.

A ketone of the general formula (II) may be obtained where the radicals R⁶ and IC, independently of one another, stand for a secondary, tertiary or quaternary C atom and Q for a R⁶ and IC-bridging substituted or unsubstituted group with 1 to 10 carbon atoms.

In a further non-limiting embodiment, R⁶ and R⁷ in formula (II) and formula (I), independently of one another, stand for a secondary or tertiary carbon atom. In a quite particularly non-limiting embodiment, one of the two radicals R⁶ and R⁷ stands for a secondary carbon atom, whereas the respective other radical R⁶ or R⁷ stands for a tertiary carbon atom. The radicals R⁶ and R⁷ may represent hydrocarbon groups. Either R⁶ or R⁷, or both R⁶ and R⁷, can represent hydrocarbon groups.

In a further non-limiting embodiment Q stands for a R⁶ and R⁷-bridging substituted or unsubstituted group, wherein the R⁶ and R⁷-bridging part of Q is chosen such that a four-, five-, six-, seven- or eight-membered ring is present. Q is particularly preferred if the R⁶ and R⁷-bridging part of Q is chosen such that a five- or six-membered ring is present. Q may be a hydrocarbon group in a non-limiting embodiment.

The bridging part —R⁷-Q-R⁶— of the ketone of the general formula (II) is a hydrocarbon group in a non-limiting embodiment.

In a non-limiting embodiment, the ketone of the general formula (II) contains at least one semicyclic or exocyclic double bond. The storage of a fragrance with at least one cyclic double bond via this cyclic double bond, wherein the fragrance has at least one additional semicyclic or exocyclic double bond, represents a particular requirement, as frequently in reactions of the production process, no sufficient selectivity can be achieved between the double bonds or the fragrance is indeed stored selectively via the semicyclic or exocyclic double bond. Upon radiation with natural sunlight or a commercially available light source, these undesired isomers are cleaved, either not at all or at least not at sufficient speed, with the result that the scent impression of a multi-component perfume oil mixture can be changed decisively.

On the contrary, the method makes possible the selective storage of a fragrance with at least one cyclic double bond via the cyclic double bond thereof, regardless of the number and the chemical properties of further semicyclic or exocyclic double bonds. Additionally, the method makes possible the generation of pro-fragrances in diastereomerically-enriched and/or enantiomerically-enriched or diastereomerically-pure and/or enantiomerically-pure form, which inter alia leads to improving the efficiency and lightness of the cleaving process. In turn this produces a unified cleavage and thus fragrance profile.

In a quite particularly non-limiting embodiment, the ketone of the general formula (II) is dihydrocarvone or isodihydrocarvone, in particular (+)-dihydrocarvone or (+)-isodihydrocarvone.

The radicals R⁸ and R⁹ of the phosphonate (III) stand, in each case independently of one another, for an alkoxy group with 1 to 15 carbon atoms substituted with heteroatoms or unsubstituted. The radicals R⁸ and R⁹ of the phosphonate of the general formula (III) may be, in each case independently of one another, methoxy, ethoxy, n-propoxy or i-propoxy radicals. Phosphonates with these radicals R⁸ and R⁹ offer a good compromise of good solubility and stability of the phosphonate, low steric requirement and good reactivity. In one embodiment, the radicals R⁸ and R⁹ are identical. In one embodiment, the radicals R⁸ and R⁹ are not identical. Preferred are phosphonates in which the radicals R⁸ and R⁹, in each case independently of one another, are methoxy or ethoxy radicals. Particularly preferred are phosphonates in which R⁸ and R⁹ stand for ethoxy radicals.

The radical R¹⁰ stands for an alkyl group with 1 or 2 to 10 carbon atoms. The radical R¹⁰ of the phosphonate of the general formula (III) may be or include, but is not limited to, a methyl, ethyl, n-propyl, i-propyl, n-butyl, sec-butyl, i-butyl or t-butyl radical, wherein methyl, ethyl, n-propyl, and i-propyl radicals are preferred. Particularly preferred are phosphonates in which R¹⁰ stands for a methyl, even more preferably for an ethyl radical.

The alkyl parts of the radicals R⁸, R⁹ and R¹⁰ can be identical (e.g. R⁸ and R⁹ are ethoxy radicals and R¹⁰ is an ethyl radical). For this it is preferred if the alkyl parts of the radicals R⁸, R⁹ and R¹⁰ are all selected from the group consisting of methyl, ethyl, n-propyl, i-propyl, n-butyl, sec-butyl, i-butyl, and t-butyl, preferably consisting of methyl, ethyl, n-propyl, and i-propyl, more preferably of methyl and ethyl. Phosphonates in which the alkyl parts of the radicals R⁸, R⁹ and R¹⁰ all stand for ethyl, i.e. R⁸ and R⁹ stand for ethoxy radicals and R¹⁰ stands for an ethyl radical, are particularly preferred.

The 1,2-reduction of the alpha,beta-unsaturated ester of the general formula (IV) to the alcohol of the general formula (V) can be carried out under all conditions known to a person skilled in the art for such conversions. Preferably, this conversion takes place in the presence of an aluminum hydride-based reducing agent such as e.g. lithium-aluminum hydride (LAH), diisobutyl aluminum hydride (DIBALH), sodium-bis-(2-methoxy-ethoxy)-aluminum dihydride (Red-Al®) and so forth. Particularly preferred for the reduction of the alpha,beta-unsaturated ester of the general formula (IV) to the alcohol of the general formula (V) is the use of LAH.

The 1,2-reduction takes place in a solvent. Suitable solvents can for example be selected from the group of the essential solvents such as diethyl ether or tetrahydrofuran (THF) or dimethoxyethane (DME), halogenalkanes, aromatic solvents such as toluene or benzene or mixtures thereof, wherein THF and in particular diethyl ether particularly preferred solvents.

The temperature in the 1,2-reduction of the alpha,beta-unsaturated ester of the general formula (IV) depends both on the structure of the ester and also on the reducing agent, and can be between −100° C. and 200° C., for example at approximately −78° C., −40° C., −20° C., 0° C. or room temperature. In the course of the conversion, the temperature can be increased from a starting temperature, decreased, increased and decreased again, or decreased and increased again. Conventionally, the reaction mixture is brought from a low starting temperature, e.g. of −78° C., 0° C. or room temperature, in the course of the reduction to a higher temperature, such as for example the boiling temperature of the respective solvent. The reduction may be carried out between room temperature, thus 20 to 25° C., and 110° C., such as 80° C., alternatively 50° C. Optionally, however, the reduction may be carried out at lower or higher temperatures.

The ester of the general formula (IV) is used, in respect of the reducing agent, in a ratio of from 1:20 to 20:1, such as from 1:10 to 10:1, such as from 1:5 to 5:1, such as from 1:3 to 3:1, or from 1:2 to 2:1.

If necessary, the purification of the formed allyl alcohol of the general formula (V) takes place preferably by distillation, crystallization and/or chromatographic separating methods which are known to a person skilled in the art.

In a particularly non-limiting embodiment, the 1,2-reduction is carried out according to method step d) in the presence of LAH as reducing agent and diethyl ether as solvent. Here, the ester may be brought into contact with the reducing agents at room temperature (although an exothermic reaction can lead to an increase in the reaction temperature) and the reaction mixture is brought to the boil after complete addition of the reagents.

The oxidation of the allyl alcohol of the general formula (V) to the alpha,beta-unsaturated aldehyde (enal) of the general formula (VI) (method step c)) can be carried out under standard conditions for such conversions known to a person skilled in the art. These conditions include inter alia the oxidations with activated dimethyl sulfoxide such as such as e.g. Swern oxidation, Cr-mediated oxidations such as e.g. pyridinium chlorochromate (PCC), pyridinium dichromate (PDC), and Collins reagent-mediated oxidations, Ru- or tetrapropyl ammonium perruthenate-(TPAP) mediated oxidations, hypervalent iodine reagents- or Dess-Martin-Periodinane or IBX(2-iodoxybenzoic acid)-mediated oxidations, TEMPO-based oxidations and so forth. The use of mild oxidizing agents which are selective for the oxidation of allyl alcohols. In a non-limiting embodiment, manganese (II) oxide is used as oxidizing agent.

The oxidation of allyl alcohol of the general formula (V) takes place in a solvent in a non-limiting embodiment. Suitable solvents depend on the oxidizing agent used and are known to a person skilled in the art from the state of the art. If, for example, manganese (II) oxide is used, dichloromethane may be a solvent.

The reaction temperature may range between −100° C. and 100° C. and is correspondingly adapted to the reactivity of the individual reagents and the nature of the oxidation system. Appropriate reaction conditions for the above-named oxidation systems/oxidation methods, including temperatures, solvents, concentrations and reagent quantity ratios, are known to a person skilled in the art from the state of the art.

When using manganese (II) oxide as oxidizing agent, the allyl alcohol of the general formula (V) is used, in respect of the oxidizing agent, in a ratio of from 1:500 to 500:1, such as from 1:100 to 100:1, such as from 1:50 to 50:1, in particular of from 1:30 to 30:1, alternatively from 1:20 to 20:1. Also, a ratio of manganese (II) oxide:alcohol (V) of from 1:15 to 15:1 may be used. The oxidation may be carried out at room temperature, although temperatures up to the boiling point of the respective solvent can also be used.

The oxidation of allyl alcohols of the general formula (V) to the alpha,beta-unsaturated aldehydes (enals) of the general formula (VI) is particularly preferred, wherein manganese (II) oxide is used as oxidizing agent in a ratio of up to 500:1, 100:1, 50:1, 30:1, 20:1, or up to 15:1, in respect of the allyl alcohol and in dichloromethane as solvent, at room temperature.

If necessary, the formed alpha,beta-unsaturated aldehyde (enal) of the general formula (VI) is purified by distillation, crystallization and/or chromatographic separating methods which are likewise known to a person skilled in the art.

The two-stage conversion of the alpha,beta-unsaturated ester of the general formula (IV) to the alpha,beta-unsaturated aldehyde of the general formula (VI) can also take place by means of a single reaction step, wherein the ester (IV) is reduced directly to the aldehyde (VI). The typical reaction conditions for such direct reductions include, inter alia, diisobutyl aluminum hydride (DIBALH)-mediated reduction, using carefully controlled temperature conditions. Typical reaction conditions include the addition of the reducing agent at a low temperature, e.g. at −78° C., and maintaining the temperature in the course of the reaction at 0° C. or lower, −20° C. or lower, −40° C. or lower, −50° C. or lower, such as −70° C. or lower. The selectivity of the reduction for the aldehyde product (i.e. avoiding over-reduction to the corresponding alcohol) may also depend on the solvent used. Typically, the use of non-polar solvents such as e.g. toluene, hexane, pentane and further hydrocarbon solvents, aids the provision of the aldehyde.

The 1,4-reduction of method step d) can be achieved by using any reducing agent known to a person skilled in the art as selective agent for the 1,4-reduction of alpha,beta-unsaturated aldehydes (enals). Examples of this are, inter alia “Stryker's reagent” ([Ph₃PCuH]₆), lithium or sodium in liquid ammonia (or other suitable metal-containing solutions), and aluminum powder in NiCl₂. This chemical reaction can also be achieved by hydrosilylation, for example by Rh-catalyzed (by means of an achiral or chiral Rh catalyst) hydrosilylation, and subsequent hydrolysis of the product-silyl enol ether, or by selective hydrogenation.

Transfer hydrogenation, e.g. organocatalytic transfer hydrogenation, represents a particularly suitable method for selective 1,4-reduction of enals. A plurality of different organocatalytic transfer hydrogenation conditions for this reaction, including both non-stereoselective methods and also stereoselective methods, have been developed for this purpose. These include, inter alia, methods in which the carbonyl group of the enal is converted into an iminium ion and the C═C double bond of the enal is thus activated vis-à-vis the engagement of a hydride group. Here, both non-chiral (achiral) and also chiral catalysts can be used, wherein the chiral systems include both (i) systems in which the chirality lies in the amine components of the iminium ion and also (ii) systems in which the chirality lies in the counter ions of the iminium ion (so-called “asymmetric counterion directed catalysis” or ACDC). The above-named organocatalytic transfer hydrogenation methods and in particular the methods described under (i) and (ii) are particularly suitable for method step d).

Methods which are suitable for method step d) include, for example, iminium ion methods which use non-chiral (achiral) catalysts. These include transfer hydrogenation by mean of the use of a dialkyl ammonium salt in the presence of a hydride source, e.g. a Hantzsch ester. Particularly suitable dialkyl ammonium salts for this reaction are, inter alia, dibenzyl ammonium salts such as e.g. dibenzyl ammonium trifluoroacetate or dibenzyl ammonium trifluoromethane sulfonate (dibenzyl ammonium triflate), pyrrolidinium salts, morpholinium salts, piperidinium salts, dimethyl ammonium salts and N,O-dimethyl hydroxyl ammonium salts. Suitable counterions of these salts are, inter alia, halides including chloride, bromide and iodide, trifluoroacetate, triflate and so forth. Any Hantzsch ester can be used which is suitable for reductions. Particularly suitable Hantzsch esters include the following:

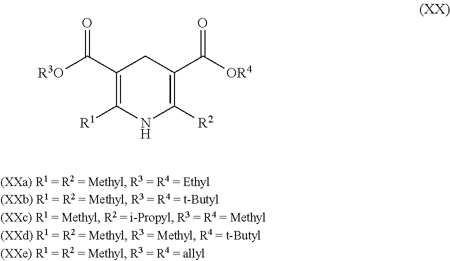

When using an achiral catalyst in method step d), the use of Hantzsch ester (XXa), together with either dibenzyl ammonium trifluoroacetate or dibenzyl ammonium triflate is particularly preferred.

The use of corresponding organic catalysts offers, inter alia, the advantage that no toxic and/or expensive chemicals, in particular heavy metals, are used in this reducing step.

Further methods which are suitable for method step d) include, for example, iminium ion-mediated methods which use chiral catalysts. These can lead to the aldehydes of the formula (VII) being obtained in diastereomerically- and/or enantiomerically-enriched form or diastereomer- and/or enantiomer-pure form.

These methods include transfer hydrogenation by means of the use of a chiral imidazolidinone in the presence of a hydride source, e.g. a Hantzsch ester. The following imidazolidinones (XXIa1/2) to (XXIe1/2)

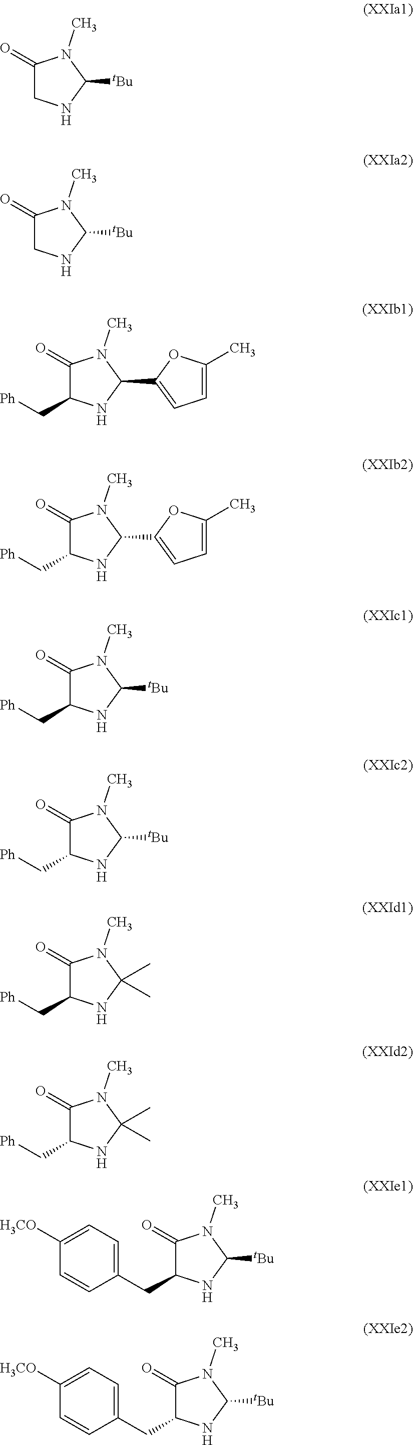

may represent suitable catalysts. These could be used as the free bases or the corresponding salts thereof, such as e.g. hydrochlorides, trifluoroacetates, dichloroacetates and so forth. Each of the above Hantzsch ester (XXa) to (XXe) can be used with one of the imidazolidinones (XXIa1/2) to (XXIe1/2) in order to carry out the reducing step d) of the present method. Particularly suitable imidazolidinones are (XXIc1)/(XXIc2) and (XXIa1)/(XXIa2). Particularly suitable Hantzsch ester are (XXa) and (XXc). Non-limiting imidazolidinone/Hantzsch ester combinations are (XXIa1)/(XXIa2) with (XXa), and (XXIc1)/(XXIc2) with (XXc). Whether e.g. imidazolinone (XXIa1) or (XXIa2) is used depends on the desired configuration of the stereocenter resulting from the reduction.

The ACDC catalysts which can be used may be or include, inter alia, chiral, phosphoric acids such as for example phosphoric acids (XXIIa) and (XXIIb). These catalysts can be used in method step d), together with one of the Hantzsch esters (XXa) to (XXe), such as either with Hantzsch ester (XXa) or (XXc), in order to carry out the targeted reduction stereoselectively. The other enantiomer (not represented) of phosphoric acid can also be used if the other product isomer/product epimer is desired.

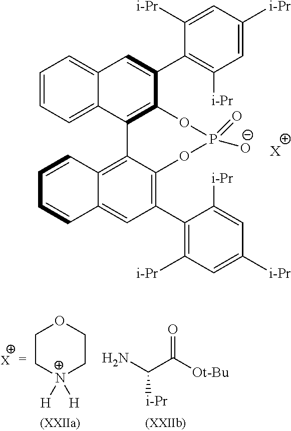

Depending on the specific structure of the ketone of the general formula (II), the use of achiral reducing agents in method step d) can, however, lead to the introduction of the hydride in a stereoselective manner. In this respect, in particular sterically hindered reducing agents are used.

The use of such stereoselective reducing methods can ultimately lead to the obtaining of chiral (diastereomerically and/or enantiomerically enriched or diastereomer- and/or enantiomer-pure) pro-fragrances and the above-mentioned advantages.

The precise reaction conditions, including temperature, solvent, concentrations and quantity ratios of the reagents are adapted according to the individual reagents, catalysts and the chosen reduction system. Typical reaction conditions for each of the above-named reductions or organocatalytic reductions are described in the state of the art and are correspondingly used with the systems. Typical reaction conditions for the described reductions are to be found for example in “March's Advanced Organic Chemistry: Reactions, Mechanisms, and Structure, 2013, 7th Edition, Michael B. Smith (ISBN-13: 978-0470462591)” and documents cited therein. In respect of the reaction conditions of the above-mentioned organocatalytic reductions, inter alia “Enantioselective Organocatalyzed Reactions I: Enantioselective Oxidation, Reduction, Functionalization and Desymmetrization, 2011, Springer, Rainer Mahrwald, (ISBN-13: 978-9048138647)” and in particular chapter 2 of “Asymmetric Hydrogenations Using Hantzsch ester,” and documents cited therein, contain specific information in respect of the typical conditions which are to be used.

The imidazolidinone (XXIa1) or (XXIa2) may be used together with Hantzsch ester (XXa) when converting the enal of the general formula (VI) to the aldehyde of the general formula (VII). Preferably, this is carried out in a solvent. Suitable solvents are, inter alia, halogen alkane solvents such as for example dichloromethane, in particular chloroform.

The reaction temperature between −100° C. and 100° C. and is correspondingly adapted to the reactivity of the individual reagents and the desired selectivity. In so doing, the temperature of the reaction mixture can for example initially be a low temperature (e.g. between −78° C. and −30° C.) and be increased, stepwise, in the course of the reaction to for example −20° C., −10° C., 0° C., or room temperature, thus 20 to 25° C. Preferably, the temperature of the reaction mixture in the course of the reaction is maintained between 0° C. and −78° C., preferably between −10° C. and −50° C., more preferably between −20° C. and −40° C., even more preferably between −25° C. and −35° C., in particular at −30±3° C.

In respect of the enal of the general formula (VI), the imidazolidinone is used in preferably 1 mol % or more to 100 mol % or less, preferably 5 mol % or more to 50 mol % or less, more preferably 7.5 mol % or more to 40 mol % or less, even more preferably 10 mol % or more to 30 mol % or less, in particular 15 mol % or more to 25 mol % or less. Particularly preferably, the use of the imidazolidinone is in a quantity of 20±3 mol %.

In respect of the enal of the general formula (VI), the Hantzsch ester is used in a ratio of from preferably 1:20 to 20:1, preferably of from 1:10 to 10:1, more preferably of from 1:5 to 5:1, even more preferably of from 1:2 to 2:1, in particular of from 1:1.5 to 1.5:1. Particularly preferably, the use of the Hantzsch ester is in 120±20 mol % in respect of the enal of the general formula (VI).

When converting the enal of the general formula (VI) to the aldehyde of the general formula (VII), the use of the imidazolidinone (XXIa1)/(XXIa2) in a quantity of 20 mol % and of the Hantzsch ester (XXa) in a quantity of 120 mol % (both in relation to the starting enal), is particularly preferred. For this, preferably chloroform is used as solvent, and the reaction is carried out at a temperature between −20° C. and −40° C., preferably at a temperature of −30±3° C.

If necessary, the purification of the formed aldehyde of the general formula (VII) takes place preferably by distillation, crystallization and/or chromatographic separating methods which are known to a person skilled in the art.

In step e) of the method, the aldehyde obtained under step d) of the general formula (VII) is converted to an alcohol of the general formula (VIII).

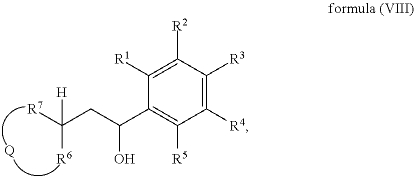

This is preferably an addition reaction, in particular an addition of a nucleophile to the aldehyde of the general formula (VII).

If method step e) is an addition of a nucleophile to the aldehyde of the general formula (VII), preferably a halide according to general formula (IX) transformed into a metal organyl is used,

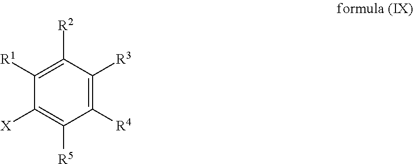

wherein R¹ to R⁵ are as defined above and X stands for —F, —Cl, —Br, or —I. After the transformation of the halide of the formula (IX) into the metal organyl, subsequently the metal organyl is brought to react with the aldehyde of the formula (VII). If one or more of the radicals R¹ to R⁵ are also halogen, X must correspondingly be selected from —F, —Cl, —Br, or —I, with the result that the metal organyl can be formed at the desired, X-bearing position of the aromatic ring.

Preferably, the radicals R¹ to R⁵ stand, in each case independently of one another, for hydrogen, methyl, isopropyl or methoxy groups, wherein methoxy groups are particularly preferred. Halobenzenes with these radicals R¹ to R⁵ are either commercially available or synthetically easily accessible, and influence the absorption spectrum of the pro-fragrance advantageously. Thus, by a suitable choice, the radicals R¹ to R⁵, the release speed of the stored fragrance can be increased or reduced upon radiation with preferably natural sunlight or commercially available light sources.

Four of the five aryl substitutes R¹, R², R³, R⁴ and R⁵ can stand simultaneously for hydrogen. Preferably, R¹, R², R⁴ and R⁵ stand for hydrogen. If R¹, R², R⁴ and R⁵ stand for hydrogen, the substitute in para position R³ preferably stands for a halogen atom, in particular —F, —Cl, —Br or —I, NO₂, a linear or branched, substituted or unsubstituted alkoxy group with up to 15 C atoms or a linear or branched, substituted or unsubstituted alkyl group with up to 15 C atoms.

In a particularly non-limiting embodiment, R³ stands for —Cl, —Br, —NO₂ or an alkyl or alkoxy group comprising up to 4 C atoms. Preferably, the linear or branched, substituted or unsubstituted alkyl group is a methyl or ethyl group and/or the linear or branched, substituted or unsubstituted alkoxy group is a methoxy, ethoxy, isopropoxy or tert-butoxy group, wherein a methoxy group is quite particularly preferred.

A substitution in para position R³ is particularly preferred, as the electronic structure of the aromatic ring can be most effectively modified here, as a result of which the absorption maximum of a ketone of the general formula (I) can be adapted to a specific wavelength easily.

Particularly preferred are halides of the formula (IX) and ketones of the formula (I), in which R¹ to R⁵ all stand for hydrogen.

Preferably, the metal of the metal organyl is selected from the group Mg, Zn, Ce, and Li. Particularly preferred are metal organyls in which the metal is Mg. If the metal of the metal organyl is Mg, the metal organyl can be formed via the usual methods for forming Grignard reagents known to a person skilled in the art. These include, inter alia, the direct reaction of a halobenzene with Mg and the so-called “halogen-Mg exchange.” In the “halogen-Mg exchange,” a pre-formed Grignard reagent is reacted with the halobenzene and thereby, the halogen atom of the benzene ring is replaced by the Mg atom of the Grignard reagent. A typical Grignard reagent is isopropyl magnesium chloride. Reaction conditions for the formation of Grignard reagents are known from the state of the art.

In some cases, the metal organyl compounds which are reacted with the aldehyde of the general formula (VII) are commercially available. For this, the above-mentioned transformation of a halide of the formula (IX) into the desired metal organyl can be dispensed with, as the metal organyl is already available, per se.

In the above-named addition of a nucleophile to an aldehyde of the general formula (VII), the reaction conditions, including temperatures, solvents, concentrations and reagent quantity ratios are adapted, corresponding to the reactivity of the individual reagents and the nature of the specific reaction system. Typical reaction conditions for each of the above-mentioned reaction systems (e.g. systems in which Li, Zn, Ce or Mg is used as metal) are known, from the state of the art, to a person skilled in the art.

Particularly preferred, is the conversion of aldehydes of the general formula (VII) to alcohols of the general formula (VIII) by reaction with a corresponding Grignard reagent. The Grignard reagent can either be generated as described above, or purchased.

Preferably, this conversion is carried out in a solvent. Suitable solvents are inter alia hydrocarbon solvents such as toluene, cyclohexane and essential solvents such as diethyl ether, tetrahydrofuran (THF), dimethoxy ethane (DME), or mixtures thereof. Particularly preferred is diethyl ether.

The temperature upon conversion of aldehydes of the general formula (VII) to alcohols of the general formula (VIII) by reaction with a corresponding Grignard reagent can be between −100° C. and 200° C. In the course of the conversion, the temperature can be increased from a starting temperature, decreased, increased and decreased again, or decreased and increased again. In the formation of a Grignard reagent from Mg and a corresponding halide, the reaction mixture is typically brought to reflux and maintained at this temperature until formation of the Grignard reagent was complete. During the reaction of an aldehyde of the general formula (VII) with the Grignard reagent, conventionally a low starting temperature (e.g. of −78° C., 0° C. or room temperature) is used, and in the course of the reaction, the temperature is for example increased to the boiling point of the respective solvent. The reaction is preferably carried out between room temperature, thus 20 to 25° C., and 110° C., preferably between room temperature and 85° C., more preferably between room temperature and 50° C. Optionally, however, it may be preferred to carry out the reaction at lower or higher temperatures.

In respect of the aldehyde of the general formula (VII), the Grignard reagent is used in a ratio of from preferably 1:20 to 20:1, preferably of from 1:10 to 10:1, more preferably of from 1:5 to 5:1, even more preferably of from 1:3 to 3:1, in particular of from 1:2 to 2:1. Particularly preferred is the use of 200 mol % of the Grignard reagent (in respect of 100 mol % of the aldehyde of the general formula (VII)).

The use of a Grignard reagent, in particular of a Grignard reagent in which R¹ to R⁵ stand for hydrogen, may be used in a ratio of 2:1, in respect of the aldehyde of the general formula (VII). Preferably, here, diethyl ether is used as solvent and the reaction is carried out after formation of the Grignard reagent at the boiling point of the solvent.

If necessary, the purification of the formed alcohol of the general formula (VIII) takes place preferably by distillation, crystallization and/or chromatographic separating methods which are known to a person skilled in the art.

The oxidation of the benzyl alcohol of the general formula (VIII) to the ketone of the general formula (I) in method step f) can be carried out under standard conditions for such conversions, known to a person skilled in the art. These may include inter alia the oxidation methods named further above for method step c). For this, a catalytic quantity of an oxidizing agent, such as for example Cr(III) oxide in the presence of a Co oxidizing agent, such as for example tert-butyl hydroperoxide (tBuOOH), is suitable.

The oxidation of the benzyl alcohol of the general formula (VII) takes place preferably in a solvent. Suitable solvents depend on the oxidizing agent used and are known to a person skilled in the art from the state of the art. If, for example, tBuOOH is used in the presence of a catalytic quantity of CrO₃, dichloromethane represents a particularly preferred solvent.

The reaction temperature is preferably between −100° C. and 100° C. and is correspondingly adapted to the reactivity of the individual reagents and the nature of the oxidation system. For the above-named oxidations, appropriate reaction conditions including temperatures, solvents, concentrations and reagent quantity ratios are known to a person skilled in the art from the state of the art. When using tBuOOH in the presence of a catalytic quantity of CrO₃, room temperature represents a particularly suitable reaction temperature.

When using a catalytic quantity of CrO₃ in the presence of the Co oxidizing agent tert-butyl hydroperoxide (tBuOOH), in respect of the benzyl alcohol of the general formula (VII), the CrO₃ is used preferably in a quantity of from 0.01 mol % or more to 95 mol % or less, preferably of from 0.1 mol % or more to 50 mol % or less, more preferably of from 0.5 mol % or more to 20 mol % or less, even more preferably of from 1.0 mol % or more to 10 mol % or less, in particular of from 2 mol % or more to 8 mol % or less. Particularly preferred is the use of CrO₃ in a quantity of 5±2 mol %. In respect of the benzyl alcohol of the general formula (VII), the tert-butyl hydroperoxide is used in a ratio of from preferably 1:100 to 100:1, preferably of from 1:50 to 50:1, more preferably of from 1:30 to 30:1, even more preferably of from 1:20 to 20:1, in particular of from 1:10 to 10:1. Particularly preferred is the use of the tert-butyl hydroperoxide in a quantity of 600±200 mol %, in respect of the benzyl alcohol (VII).

Particularly preferred is the oxidation of benzyl alcohols of the general formula (VII) to ketones of the general formula (I), wherein 5 mol % CrO₃ is used in the presence of 600 mol % tert-butyl hydroperoxide (both in respect of 100 mol % of the benzyl alcohol (VII)). For this, the use of dichloromethane as solvent is particularly suitable. The reaction is carried out preferably at room temperature.

If necessary, the formed alpha,beta-unsaturated aldehyde (enal) of the general formula (VI) is purified preferably by distillation, crystallization and/or chromatographic separating methods which are known to a person skilled in the art.

In the ketones, a substitution in para position R³ is particularly preferred as the electronic structure of the aromatic ring can be most effectively modified here, whereby the absorption maximum of a ketone of the general formula (I) can be adapted to a specific wavelength easily.

Likewise preferred is a ketone of the general formula (I), in which R¹, R², R³, R⁴ and R⁵ stand for hydrogen.

A diastereomerically- or enantiomerically-enriched substance has an excess of a specific diastereomer or enantiomer if the percentage of the diastereomer or enantiomer present in excess is of more than 50%, up to 100% (diastereomer- or enantiomer-pure). In reactions which result in a new stereocenter, the epimer ratio of the newly-produced stereocenter of the resultant diastereomers or enantiomers (depending on the structure of the starting material of the reaction) can be between 1:1 and 0:1 (i.e. 100% of one epimer). If a method step, such as for example step d), is carried out stereoselectively (e.g. by means of the use of a chiral catalyst or a chiral imidazolidinone or an ammonium salt with a chiral counterion), the epimer ratio of the newly-created stereocenter is preferably at least 2:1 or 3:1, preferably at least 5:1, more preferably at least 9:1, particularly preferably at least 95:5, in particular at least 99:1.

A ketone of the general formula (XII) or (XIII) may be obtained, which, by reacting (+)-dihydrocarvone or (+)-isodihydrocarvone with a phosphonate of the general formula (III) in method step a) and subsequent stereoselective reduction by means of an achiral, preferably a chiral catalyst in method step d).

Ketones of the general formula (I) were not able to be produced selectively thus far, but are particularly interesting from an olfactory point of view, as the stored fragrance is limonene. Limonene is one of the most important base components for multicomponent perfume oil mixtures and gives the consumer a fresh scent impression which is associated with purity.

An agent containing at least one compound of the general formula (X) or (XI) may be obtained,

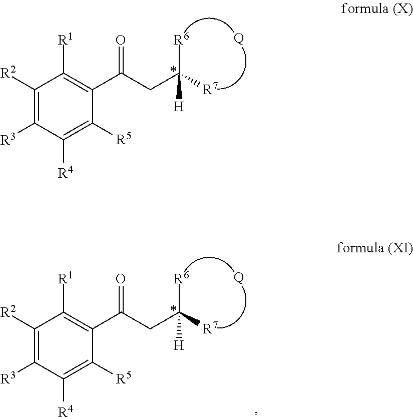

by the method, wherein R¹ to R⁷ and Q are as defined above.

In a non-limiting embodiment, the agent is a washing agent or cleaning agent, cosmetic agent, adhesive or a printing ink, wherein washing agents or cleaning agents and cosmetic agents are particularly preferred.

In a further non-limiting embodiment, such an agent contains a ketone of the general formula (X) and/or (XI) in a total quantity between 0.0001 and 10 wt.-%, advantageously between 0.0005 and 5 wt.-%, more advantageously between 0.001 and 3 wt.-%, in particular between 0.005 and 2 wt.-%, wt.-% in each case relative to the whole agent.

In a non-limiting embodiment, an agent containing a ketone of the general formula (X) or (XI), such as e.g. washing agent or cleaning agent, cosmetic agent, adhesive or printing ink, contains at least one further fragrance. The preferably used fragrances or perfume oils are not subjected to any limitations whatsoever. Thus, preferably synthetic or natural fragrance compounds of the ester, ether, aldehyde (fragrance aldehydes) ketone (fragrance ketones), alcohol, hydrocarbon, acid, hydrocarbon ester, aromatic hydrocarbon, aliphatic hydrocarbon, saturated and/or unsaturated hydrocarbon type and mixtures thereof can be used as fragrances.

As fragrance aldehydes or fragrance ketones, all conventional fragrance aldehydes can be used which are typically used to evoke a pleasant fragrance sensation. Suitable fragrance aldehydes and fragrance ketones are generally known to a person skilled in the art. The fragrance ketones can include all ketones which can endow a desired fragrance or freshness sensation. Mixtures of different ketones can also be used Ketones which can be used are e.g. alpha-damascone, delta-damascone, iso-damascone, carvone, gamma methyl ionone, iso E super, 2,4,4,7-tetramethyl-oct-6-ene-3-one, benzyl acetone, beta damascone, damascenone, methyl dihydrojasmonate, methyl cedrylone, hedione and mixtures thereof. Suitable fragrance aldehydes can be any aldehydes which give a desired fragrance or freshness sensation according to the fragrance ketones. These can in turn be individual aldehydes or aldehyde mixtures. Suitable aldehydes are for example melonal, triplal, ligustral, adoxal, lilial and so forth. The fragrance aldehydes and fragrance ketones can have an aliphatic, cycloaliphatic, aromatic, ethylenically unsaturated structure or a combination of these structures. Moreover, further heteroatoms or polycyclic structures may be present. The structures can have suitable substituents such as hydroxyl or amino groups. Suitable fragrances of the ester type are for example benzyl acetate, phenoxyethyl isobutyrate, p-tert-butyl cyclohexyl acetate and so forth. Fragrance compounds of the hydrocarbon type are for example terpenes such as limonene and pinene. Suitable fragrances of the ether type are for example benzyl ethyl ether and ambroxan. Suitable fragrance alcohols are for example 10-undecene-1-ol, 2,6-dimethylheptane-2-ol, 2-methylbutanol, 2-methylpentanol, 2-phenoxyethanol, 2-phenylpropanol and so forth. Fragrances or perfume oils can also be natural fragrance mixtures as are accessible from plant sources. The fragrances or perfume oils can also be essential oils such as for example angelica root oil, anise oil, arnica blossom oil and so forth. The total quantity of the at least one further fragrance in the agent, such as for example washing agent or cleaning agent, cosmetic agent, adhesive or printing ink, is preferably between 0.001 and 5 wt.-%, advantageously between 0.01 and 4 wt.-%, more advantageously between 0.1 and 3 wt.-% and quite particularly preferably between 0.5 and 2 wt.-%, relative to the total agent. Preferably, mixtures of different fragrances from the different above-named fragrance classes are used which collectively produce an attractive fragrance note.

In a non-limiting embodiment, the agents, such as e.g. washing agents or cleaning agents, cosmetic agents, adhesives or printing inks, contain at least one surfactant, preferably selected from the group consisting of anionic, cationic, non-ionic, zwitterionic, amphoteric surfactants or mixtures thereof.

A preferred agent can be solid or liquid, wherein liquid agents, in particular washing agents or cleaning agents or laundry auxiliaries, are preferred. In particular for the case that the agent is a washing agent or cleaning agent, it is preferred that it contains at least one surfactant selected from the group consisting of anionic, non-ionic, zwitterionic and amphoteric surfactants. In particular for the case that the agent is a plasticizing agent (“2in1”), it is preferred that it contains a plasticizing component as well as at least one surfactant selected from the group consisting of anionic, non-ionic, zwitterionic and amphoteric surfactants. Laundry auxiliaries are used for selective laundry pre-treatment before washing where stains or strong dirt marks occur. The laundry auxiliaries include for example pre-treatment agents, softening agents, color removers and bleaching agents.

In particular if the agent is a softener, it is preferred that it contains a plasticizing component. Softeners are preferred as agents as they come into contact with the textiles only in the last step of a conventional textile washing program, and thus as large a quantity as possible of the fragrances can adhere to the textiles without the risk of the fragrances being removed again in subsequent steps. It is quite particularly preferred that the plasticizing component is an alkylated, quaternary ammonium compound, wherein at least one alkyl chain is interrupted by an ester or amido group. The plasticizing component comprises for example quaternary ammonium compounds such as monoalk(en)yl trimethyl ammonium compounds, dialk(en)yl dimethyl ammonium-compounds, mono, di or triesters of fatty acids with alkanol amines. Quaternized protein hydrolyzates or protonated amines represent further plasticizing components which can be used. Furthermore, cationic polymers are suitable plasticizing components. Polyquaternized polymers (e.g. Luviquat® Care from BASF) and also cationic biopolymers based on chitin, and derivatives thereof, for example the polymer which can be obtained under the trade name Chitosan® (manufacturer: Cognis) can likewise be used. Further suitable plasticizing components include protonated or quaternized polymers. Particularly preferred plasticizing components are alkylated quaternary ammonium compounds, where at least one alkyl chain is interrupted by an ester group and/or an amido group. N-methyl-N-(2-hydroxy-ethyl)-N,N-(ditalgacyloxyethyl)ammonium methosulfate or bis-(palmitoyloxyethyl)-hydroxyethyl methylammonium methosulfate are quite particularly preferred.

The agent, in particular in the form of softeners, can also contain non-ionic plasticizing components, such as above all polyoxyalkylene glycerol alkanoates, polybutylenes, long-chained fatty acids, ethoxylated fatty acid ethanolamides, alkylpolyglucosides, in particular sorbitan mono-, di- and triesters, and fatty acid esters of polycarboxylic acids. Advantageously, the softener contains, as agent, the plasticizing component in quantities of from 0.1 to 80 wt.-%, conventionally 1 to 40 wt.-%, preferably 2 to 20 wt.-% and in particular 3 to 15 wt.-% and the at least one fragrance or the mixture of different fragrances in quantities of from advantageously 0.1 to 20 wt.-%, preferably 1 to 13 wt.-% and in particular 2 to 8 wt.-%, in each case relative to the total quantity of the agent.

As further component, the agent can optionally contain one or more non-ionic surfactants, wherein surfactants which are usually also used in washing agents can be used.

It is furthermore preferred that the agent, in particular in the form of a washing agent or cleaning agent, additionally contains further advantageous ingredients which are known to a person skilled in the art. Thus the agent, such as in particular washing agent or cleaning agent, can contain further ingredients in addition to the surfactants and/or plasticizing compounds, which ingredients further improve the practical and/or aesthetic properties of the agent. Non-limiting agents may include or contain, in addition to one or more substances from the group of builders, bleaching agents, bleach catalysts, bleach activators, enzymes, electrolytes, non-aqueous solvents, pH adjusters, fluorescing agents, dyes, hydrotropes, suds suppressors, silicon oils, anti redeposition agents, optical brighteners, graying inhibitors, run-in preventers, crease protection agents, dye transfer inhibitors, antimicrobial active ingredients, germicides, fungicides, antioxidants, preservatives, corrosion inhibitors, antistatic agents, bitters, ironing aids, repellents and impregnating agents, swelling agents and non-slip agents, neutral filler salts and UV absorbers. As builders which can be contained in the agents, there are in particular to be named also silicates, aluminosilicates such as in particular zeolites, carbonates, salts of organic di- and polycarboxylic acids and mixtures of these substances.

As cleaning agent, the agent can be used e.g. for cleaning hard surfaces. For example, these can be agents for cleaning crockery, which are used for manual or machine cleaning of crockery. They can also be conventional industrial or household cleaners, with which hard surfaces such as furniture surfaces, ceramics, tiles, wall and floor coverings can be cleaned. Hard surfaces include, in addition to crockery, also all remaining hard surfaces, in particular those made of glass, ceramic, plastic, wood or metal, domestically and in industry. As with all other agents, these can be solid or liquid formulations, wherein solid formulations can be present as powder, granulate, extrudate, in tabletted form, as tablets or as compressed and/or melted shaped bodies. Liquid formulations can be solutions, emulsions, dispersions, suspensions, microemulsions, gels or pastes.

The production of solid agents, i.e. washing agents or cleaning agents, does not present any difficulties and can in principle take place in known manner or by spray-drying or granulation, wherein optional peracid compounds and optional bleach catalysts can, if necessary, be added later. To produce agents with an increased bulk density, in particular in the range of from 650 g/L to 950 g/L, a method comprising an extrusion step is preferred. The production of liquid agents likewise also does not present any difficulties, and can likewise take place in known manner. The pro-fragrances can be incorporated in particular together with other fragrances.

The use of a ketone of the general formula (X) or (XI) may occur, which can be produced by the method as a washing agent or cleaning agent, cosmetic agent, adhesive or a printing ink for prolonging the scent impression of the washing agent or cleaning agent, cosmetic agent, adhesive or printing ink. By using the ketone of the general formula (X) or (XI) in an agent described above, a possibly unpleasant odor of other ingredients of an agent described above is effectively masked over a long period of time.

The use of a ketone of the general formula (X) or (XI) may occur, which can be produced by the method for prolonging the scent impression on a surface treated with a washing agent or cleaning agent, cosmetic agent, adhesive or printing ink. On a surface treated with a ketone of the general formula (X) or (XI), or another soft surface, the ketone produced a scent impression which consumers perceive to be pleasant and fresh, which scent impression is associated with cleanliness and purity, in particular in washing agents or cleaning agents.

It should be understood that the use of protective groups in the syntheses described herein may be desired and/or necessary for specific embodiments. Examples of protective groups and methods for the removal thereof are described in the following reference work: “Greene's Protective Groups in Organic Synthesis,” Peter G. M. Wuts, Wiley, 5^{th} Edition, Oct. 27, 2014 (ISBN-13: 000-1118057481).

Reaction conditions for chemical conversions which are described above as “known to a person skilled in the art,” “standard conditions” or similar, can be found inter alia in “March's Advanced Organic Chemistry: Reactions, Mechanisms, and Structure, 2013, 7th Edition, Michael B. Smith (ISBN-13: 978-0470462591)” and documents cited therein.

All above-named non-limiting embodiments, or the respective described features, can also individually be combined with one another. Additionally, the term “comprising” also covers the alternatives in which the products/methods/uses with respect to which the term “comprising” is used, exclusively for the elements described subsequently.

### EXAMPLES

Unless otherwise established, all processes and parameter measurements have been carried out at room temperature (20 to 25° C.).

Production of ethyl[(2R,5R)-5-isopropenyl-2-methyl cyclohexylidene] acetate

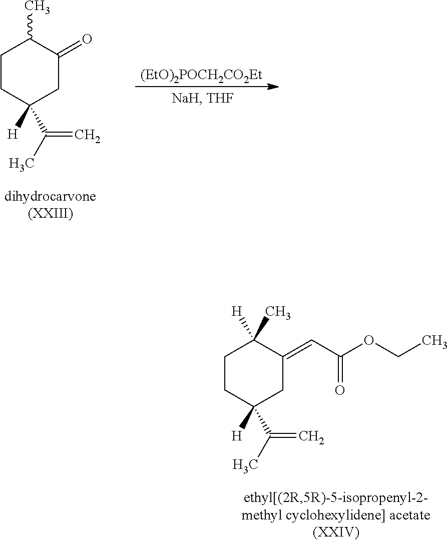 [SMILES: "(EtO)2POCH2CO2Et" ; C=C(C)[C@@H]1CCC(C)C(=O)C1 ; C=C(C)[C@@H]1CC[C@@H](C)/C(=C/C(=O)OCC)C1]

2.64 g (110 mmol, 1.5 eq) sodium hydride was suspended in 85 mL THF and cooled to 0° C. To this was added, dropwise, 24.7 mL (125 mmol, 1.7 eq) triethyl phosphonoacetate in 17 mL THF such that gas formation remained small, and the mixture foamed only slightly. After this addition, stirring took place for 30 minutes at room temperature, followed by renewed cooling to 0° C. and dropwise addition of 12 mL (73 mmol, 1 eq) dihydrocarvone (XXIII) (mixture of (+)-dihydrocarvone and (+)-isodihydrocarvone; commercially available from, inter alia, Sigma-Aldrich) in 11 mL THF. The mixture was then stirred overnight at room temperature. Finally, 50 mL saturated ammonium chloride solution was added, the phases were separated, the aqueous phase was extracted twice with 50 mL diethyl ether each time, the combined organic phases washed with saturated sodium chloride solution, dried over magnesium sulfate and the solvent removed in the vacuum.

The raw product (21.4 g) was further used for the following reaction step without being worked up.

¹H-NMR (CDCl₃, 600 MHz): δ [ppm]=5.53 (s, 1×H at C4), 4.67 (m, 2×H at C5), 4.08 (m, 2×H C6), 2.29-1.63 (m, 8×H at C7-C11), 1.68 (s, 3×H at C14), 1.22 (m, 3×H at C12), 1.09 (d, J=7.2 Hz, 3×H at C13).

¹³C-NMR (CDCl₃, 150 MHz): δ [ppm]=172.0 (C1), 166.4 (C2), 149.1 (C3), 110.5 (C4), 108.9 (C5), 59.4 (C6), 47.2 (C7), 38.1 (C8), 32.5 (C9), 30.1 (C10), 29.3 (C11), 20.6 (C12), 18.3 (C13), 14.3 (C14).

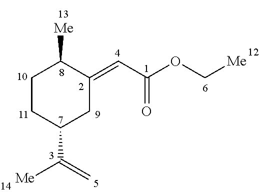 [SMILES: C=C(C)[C@@H]1CC[C@@H](C)/C(=C/C(=O)OCC)C1]

MS (EtOAc, EI): m/z [%]=222 [M⁺] (39%), 193 (51%), 179 (100%), 177 (69%), 176 (78%), 161 (31%), 151 (44%), 149 (59%), 148 (65%), 147 (33%), 133 (80%), 121 (36%), 119 (51%), 107 (80%), 105 (76%), 93 (70%), 91 (78%), 81 (32%), 79 (52%), 77 (39%), 67 (32%).

### Synthesis of 2-[(2R,5R)-5-isopropenyl-2-methyl cyclohexylidene] ethanol

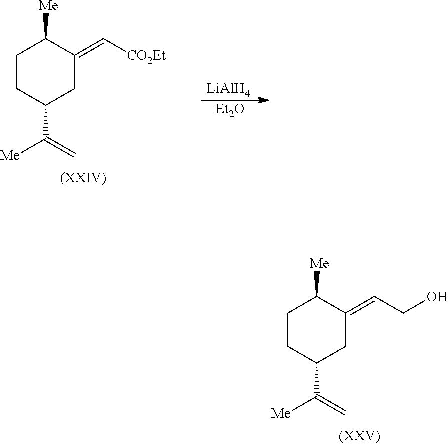

11.6 g (305.8 mmol, 2 eq) lithium aluminum hydride was suspended in 305 mL diethyl ether (c=1 mol/L) and 34.0 g (152.9 mmol, 1 eq) ethyl[(2R,5R)-5-isopropenyl-2-methyl cyclohexylidene]acetate (XXIV) in 61 mL ether (c=2.5 mol/L) was added dropwise, accompanied by stirring, such that the mixture boiled slightly. Subsequently, heating to reflux took place for an hour. Thereafter, accompanied by strong stirring and cooling in the ice bath, saturated sodium sulfate solution was added dropwise until it was no longer possible to observe the development of any more hydrogen. Then, sulfuric acid (c=2.5 mol/L) was added until the precipitated deposit was dissolved again. The phases were then separated, the aqueous phase was extracted three times with 100 mL ether, each of the combined organic phases washed with saturated sodium hydrogen carbonate solution and sodium chloride solution, dried over magnesium sulfate and the solvent removed in the vacuum. 18.9 g raw product was obtained. 1.0 g raw product was purified by FSC (fused silica chromatography) on silica gel with a solvent mixture of cyclohexane and ethyl acetate in the ratio of 9:1.

R_{f}=0.45

Yield: 0.9 g (5.0 mmol, 92%)

M (C₁₂H₂₀O)=180.29 g/mol

¹H-NMR (CDCl₃, 600 MHz): δ [ppm]=5.26 (t, J=6.7 Hz, 1×H at C3), 4.65 (m, 2×H at C4), 4.12 (m, 2×H C5), 2.64 (m, 2×H at C9), 2.00-1.46 (m, 6×H at C6, C7, C8, C10), 1.67 (s, 3×H at C11), 0.99 (d, J=6.6 Hz, 3×H at C12).

¹³C-NMR (CDCl₃, 150 MHz): δ [ppm]=149.7 (C1), 146.3 (C2), 118.3 (C3), 108.6 (C4), 58.5 (C5), 46.8 (C6), 37.8 (C7), 36.6 (C8), 34.6 (C9), 31.9 (C10), 20.7 (C11), 18.0 (C12).

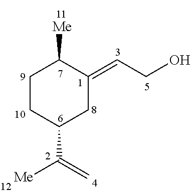

MS (EtOAc, EI): T_{R}=10.46 min; m/z [%]=180 [M⁺] (23%), 163 (22%), 162 (54%), 149 (38%), 148 (33%), 147 (75%), 137 (18%), 135 (18%), 133 (39%), 121 (41%), 120 (30%), 119 (82%), 117 (17%), 115 (19%), 109 (19%), 108 (24%), 107 (80%), 106 (30%), 105 (99%), 98 (23%), 95 (32%), 94 (27%), 93 (94%), 92 (27%), 91 (100%), 83 (20%), 81 (39%), 79 (89%), 77 (52%), 69 (23%), 67 (49%), 55 (32%), 53 (22%).

### Synthesis of 2-[(2R,5R)-5-isopropenyl-2-methyl cyclohexylidene] ethanol

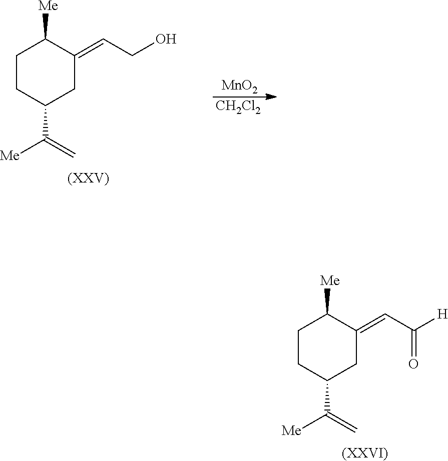

17.9 g (99.3 mmol, 1 eq) 2-[(2R,5R)-5-isopropenyl-2-methyl cyclohexylidene] ethanol (XXV) was dissolved in 99 mL dichloromethane (c=1 mol/L) and 129.5 g (1489.3 mmol, 15 eq) manganese dioxide (electrolytically deposited) added. After 64 hours' stirring at room temperature, the manganese dioxide was filtered off over Celite and the solvent removed in the vacuum. 13.3 g raw product was obtained. 3.0 g raw product was purified by FSC on silica gel with a solvent mixture of cyclohexane and ethyl acetate in the ratio of 5:1.

R_{f}=0.48

Yield: 1.8 g (10.1 mmol, 60%)

M (C₁₂H₁₈O)=178.27 g/mol

¹H-NMR (CDCl₃, 600 MHz): δ [ppm]=9.99 (d, J=8.0 Hz, 1×H at C1), 5.74 (d, J=8.0 Hz, 2×H at C5), 4.68 (s, 1×H C4), 3.37 (m, 1×H at C9), 2.15 (m, 1×H at C7), 2.04 (m, 1×H at C6), 1.94 (m, 1×H at C8), 1.90 (m, 1×H at C9), 1.79 (m, 1×H at C10), 1.67 (s, 3×H at C11), 1.42 (ddd, J=3.9, 13.0, 25.3 Hz, 1×H at C10), 1.00 (d, J=6.5 Hz, 3×H at C12)

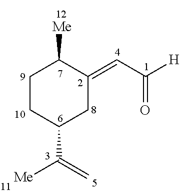

¹³C-NMR (CDCl₃, 150): δ [ppm]=190.6 (C1), 170.2 (C2), 148.3 (C3), 122.6 (C4), 109.4 (C5), 47.5 (C6), 39.4 (C7), 36.4 (C8), 35.0 (C9), 31.4 (C10), 20.5 (C11), 17.4 (C12).

MS (EtOAc, EI): T_{R}=10.62 min; m/z [%]=178 [M⁺] (100%), 163 (28%), 149 (16%), 145 (23%), 136 (17%), 135 (38%), 121 (52%), 119 (30%), 117 (23%), 107 (60%), 105 (40%), 95 (39%), 93 (65%), 92 (20%), 91 (61%), 81 (31%), 79 (50%), 77 (37%), 67 (32%), 65 (18%), 55 (18%), 53 (20%).

### Synthesis of 2-[(1S,2R,5R)-5-isopropenyl-2-methyl cyclohexyl] ethanol

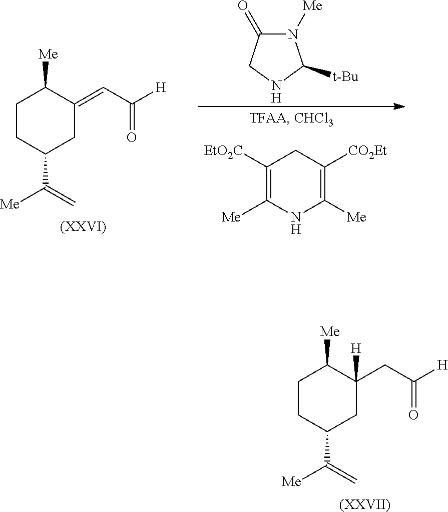

1.5 g (8.3 mmol, 1 eq) 2-[(2R,5R)-5-isopropenyl-2-methyl cyclohexylidene] ethanol (XXVI) was dissolved in 41 mL chloroform (c=0.2 mol/L) and the solution cooled to −30° C. Then, 446 mg (1.7 mmol, 0.2 eq) (S)-2-(t-butyl)-3-methyl-4-imidazolidinone trifluoroacetate and 2.5 g (9.9 mmol, 1.2 eq) diethyl-1,4-dihydro-2,6-dimethyl-3,5-pyridine dicarboxylate added, with stirring taking place for 18 hours at −30° C. Subsequently, 41 mL diethyl ether was added, with filtration over silica gel taking place, then the solvent was removed in the vacuum. The raw product was purified by FSC on silica gel with a solvent mixture of cyclohexane and ethyl acetate in the ratio of 5:1.

R_{f}=0.56

Yield: 1.2 g (6.7 mmol, 79%)

M (C₁₂H₂₀O)=180.29 g/mol

¹H-NMR (CDCl₃, 600 MHz): δ [ppm]=9.69 (dd, J=2.6, 1.3 Hz, 1×H at C1), 4.60 (d, J=5.2 Hz, 2×H at C3), 2.40 (dd, J=15.6, 4.6 Hz, 1×H C4), 2.34 (m, 1×H C8), 2.28 (ddd, J=15.6, 8.5, 2.7 Hz, 1×H at C4), 1.69-1.67 (m, 1×H at C9), 1.64 (m, 1×H at C7), 1.61 (s, 3×H at C11), 1.58 (m, 1×H at C6), 1.46-1.37 (m, 1×H at C6, 2×H at C10), 1.08 (dd, J=12.6, 3.1 Hz, 1×H at C9), 0.79 (d, J=7.0 Hz, 3×H at C12).

¹³C-NMR (CDCl₃, 150): δ [ppm]=203.0 (C1), 149.7 (C2), 108.5 (C3), 41.6 (C4), 38.5 (C5), 36.1 (C6), 34.5 (C7), 33.4 (C8), 31.3 (C9), 29.4 (C10), 20.9 (C11), 19.6 (C12).

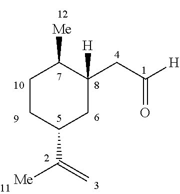

MS (EtOAc, EI): T_{R}=9.92 min; m/z [%]=180 [M⁺] (1%), 136 (100%), 121 (61%), 107 (35%), 105 (23%), 95 (20%), 93 (61%), 91 (31%), 81 (21%), 79 (41%), 77 (19%), 67 (26%), 55 (19%).

### Synthesis of 2-[(1S,2R,5R)-5-isopropenyl-2-methyl cyclohexyl]-1-phenylethanol

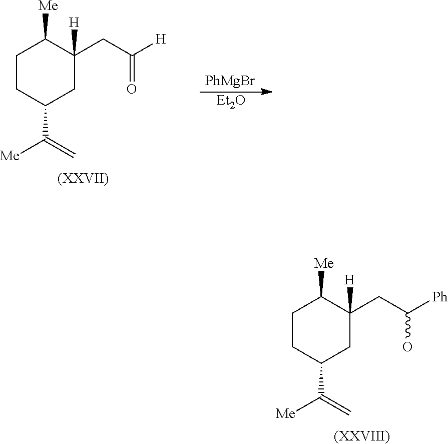

1.2 mL (11.9 mmol, 2 eq) bromobenzene in 3.0 mL diethyl ether (c=3.8 mol/L) was added to 270 mg (11.1 mmol, 2 eq) magnesium, and a drop of tetrachloromethane added. Then reflux took place until the magnesium was largely dissolved. After cooling to room temperature, 1.0 g (5.6 mmol, 1 eq) 2-[(1S,2R,5R)-5-isopropenyl-2-methyl cyclohexyl] ethanal (XXVII) in 4.0 mL ether (1.5 mol/L) was added, dropwise, and refluxed for 2 hours. Subsequently, 10 mL saturated ammonium chloride solution was added, the phases were separated, the aqueous phase was extracted twice with 50 mL ether, the combined organic phases washed with saturated sodium chloride solution, dried over magnesium sulfate and the solvent removed in the vacuum. 1.9 g of a 1:1 diastereomer mixture was obtained as raw product.

M (C₁₈H₂₆O)=258.40 g/mol

MS (EtOAc, EI): T_{R}=13.60 min; m/z [%]=258 [M⁺] (1%), 240 (42%), 149 (28%), 136 (38%), 129 (19%), 121 (23%), 109 (28%), 107 (100%), 95 (23%), 93 (26%), 91 (51%), 81 (25%), 79 (55%), 77 (41%), 67 (21%), 55 (17%).

### Synthesis of 2-[(1S,2R,5R)-5-isopropenyl-2-methyl cyclohexyl]-1-phenylethanone

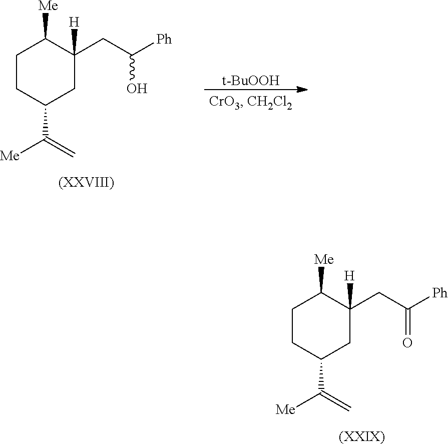

39 mg (0.4 mmol, 0.05 eq) chromium trioxide was suspended in 13 mL dichloromethane (0.6 mol/L) and 6.4 mL (46.4 mmol, 6 eq) t-butyl peroxide (70%) added. Then, 1.9 g (7.7 mmol, 1 eq) 2-[(1S,2R,5R)-5-isopropenyl-2-methyl cyclohexyl]-1-phenylethanol (XXVIII) was added, and stirred for 2 hours. Subsequently, saturated sodium sulfite solution was added until it was no longer possible to observe the development of any gas, the phases were separated, the aqueous phase was extracted twice with 40 mL diethyl ether, the combined organic phases washed with saturated sodium chloride solution, dried over magnesium sulfate and the solvent removed in the vacuum. 0.5 g of 1.4 g raw product was purified by FSC on silica gel with a solvent mixture of cyclohexane and ethyl acetate in the ratio of 50:1.

R_{f}=0.20

Yield: 178 mg (0.7 mmol, 25%)

M (C₁₈H₂₄O)=256.38 g/mol

¹H-NMR (CD₂Cl₂, 600 MHz): δ [ppm]=7.98 (d, J=7.8 Hz, 1×H at C6), 7.57 (t, J=7.4 Hz, 1×H at C4), 7.49 (t, J=7.7 Hz, 1×H C5), 4.66 (s, 2×H C7), 3.03 (dd, J=16.0, 4.5 Hz, 1×H at C9), 2.91 (dd, J=15.9, 9.4 Hz, 2×H at C10), 2.52 (m, 2×H at C11 and C12), 2.11 (t, J=11.5 Hz, 1×H at C8), 1.79-1.70 (m, 3×H at C9, C10, C13), 1.67 (s, 3×H at C15), 1.55 (m, 1×H at C14), 1.31-1.25 (m, 2×H at C13 and C14), 0.93 (d, J=6.9 Hz, 3×H at C16).

¹³C-NMR (CD₂Cl₂, 150 MHz): δ [ppm]=201.1 (C1), 151.0 (C2), 138.2 (C3), 133.3 (C4), 129.1 (C5), 128.6 (C6), 108.6 (C7), 39.3 (C8), 36.3 (C9), 36.2 (C10), 35.7 (C11), 35.6 (C12), 32.1 (C13), 30.2 (C14), 21.2 (C15), 20.0 (C16).

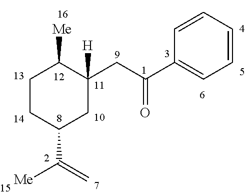 [SMILES: C=C(C)[C@@H]1CC[C@@H](C)[C@H](CC(=O)c2ccccc2)C1]

MS (EtOAc, EI): T_{R}=13.62 min; m/z [%]=256 [M⁺] (5%), 136 (100%), 121 (44%), 120 (35%), 105 (63%), 93 (25%), 91 (17%), 79 (16%), 77 (42%), 67 (10%), 55 (8%).

1. A method for producing a ketone of the general formula (I) 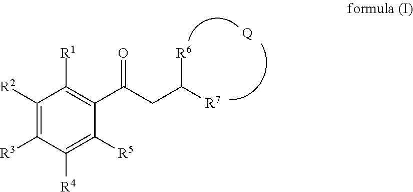
wherein R¹ to R⁵, in each case independently of one another, stand for hydrogen, halogen, NO₂—, —OH, —NR′R″R′″, or for linear, branched, or cyclic, saturated or unsaturated radicals with up to 15 carbon atoms, wherein R′, R″, and R′″, in each case independently of one another, are selected from hydrogen and up to 15 carbon atoms, wherein the carbon atoms are linear, branched, cyclic, saturated, unsaturated alkyl radicals, or combinations thereof, and wherein
the carbon atoms of the ring of formula (I) linked to R¹ and R² or R² and R³ can be a common component of an annealed five-, six- or seven-membered cycloalkyl, aryl, heteroaliphatic or heteroaryl ring, wherein the radicals R¹ and R² or R² and R³, in each case independently of one another, are completely or partly an integral component of the annealed ring; and wherein one or more hydrogen atoms, methyl groups, methylene groups, methine groups, quaternary carbon atoms, or combinations thereof of the radicals R¹ to R⁵ can, in each case independently of one another, be substituted by heteroatoms; and
wherein R⁶ to R⁷, in each case independently of one another, stand for a secondary, tertiary, or quaternary carbon atom; and wherein
Q stands for an R⁶ to R⁷-bridging substituted or unsubstituted group with up to 11 carbon atoms;
wherein the method comprises:
a) obtaining a ketone of the general formula (II) 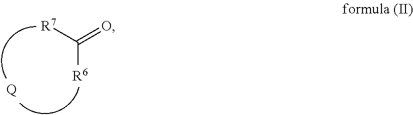
wherein the radicals R⁶, R⁷ and Q are identical to the general formula (I), with a phosphonate of the general formula (III) 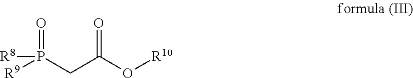
wherein R⁸ and R⁹, in each case independently of one another, stand for an alkoxy group with up to 15 carbon atoms, and R¹⁰ stands for an alkyl group with up to 10 carbon atoms, is converted to an alpha,beta-unsaturated ester of the general formula (IV) 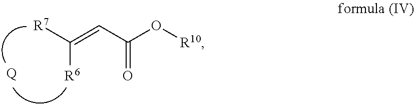
wherein one or more hydrogen atoms, methyl groups, methylene groups, methine groups, or quaternary carbon atoms of the radicals R⁸ and R⁹ can, in each case independently of one another, be substituted by heteroatoms,
b) reducing the alpha,beta-unsaturated ester of the general formula (IV) obtained in step a) to an alcohol of the general formula (V) 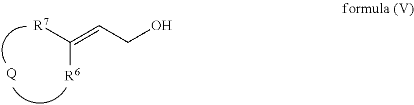
c) oxidizing the allyl alcohol of the general formula (V) obtained in step b) to an alpha,beta-unsaturated aldehyde of the general formula (VI) 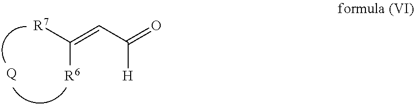
d) reducing the alpha,beta-unsaturated aldehyde of the general formula (VI) obtained in step c) to an aldehyde of the general formula (VII) 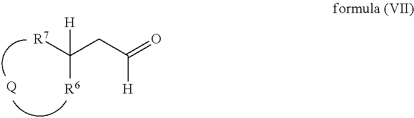
e) converting the aldehyde of the general formula (VII) obtained in step d) to an alcohol of the general formula (VIII) 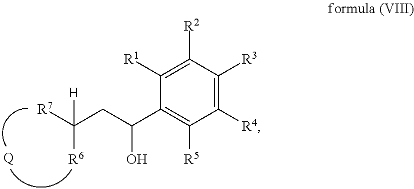
wherein the radicals R¹ to R⁵ are identical to the general formula (I) and
f) oxidizing the alcohol of the general formula (VIII) obtained in step e) to the ketone of the general formula (I).

2. The method according to claim 1, wherein the ketone of the general formula (II) has at least one semicyclic or exocyclic double bond.

3. The method according to claim 1, wherein the bridging part —R7-Q-R6 of the ketone of the general formula (II) is a hydrocarbon.

4. The method according to claim 1, wherein the ketone of the general formula (II) is selected from the group consisting of (+)-dihydrocarvone, (+)-isodihydrocarvone, (−)-dihydrocarvone, (−)-isodihydrocarvone, or mixtures thereof.

5. The method according to claim 1, wherein the radicals R⁸ and R⁹ of the phosphonate of the general formula (III), in each case independently of one another, are methoxy, ethoxy, n-propoxy, i-propoxy radicals, or combinations thereof.

6. The method according to claim 1, wherein the radical R¹⁰ of the phosphonate of the general formula (III) is a methyl, ethyl, n-propyl, i-propyl, n-butyl, sec-butyl, i-butyl, or t-butyl radical.

7. The method according to claim 1, wherein the reduction in method step d) takes place in the presence of an organic catalyst.

8. The method according to claim 1, wherein the reduction in method step d) is stereoselective.

9. The method according to claim 1, wherein the reduction in method step d) takes place in the presence of a chiral catalyst.

10. The method according to claim 1, wherein the reduction in method step d) takes place in the presence of a chiral imidazolidinone.

11. The method according to claim 1, wherein in the method step e) a halide according to the general formula (IX) is used, 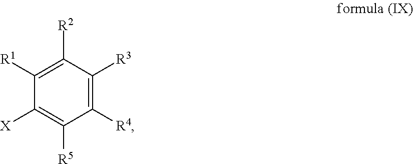
which halide is transformed into a metal organyl and then reacted with the aldehyde of the formula (VII), wherein X stands for —F, —Cl, —Br, —I and the radicals R¹ to R⁵ are as defined in claim 1 and wherein the metal is selected from the group Mg, Zn, Ce and Li.

12. The method according to claim 1, wherein the ketone of the general formula (I) is obtained with an enantiomer and/or diastereomer excess and has the structure of the formula (X) or (XI), 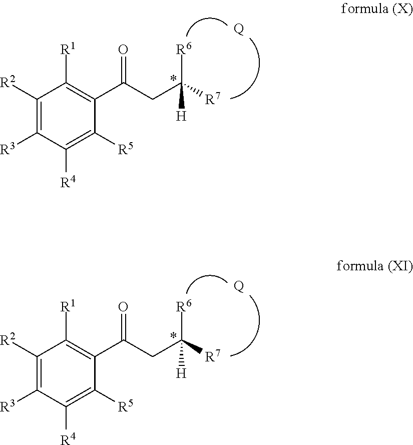
wherein R¹ to R⁷ and Q are as defined in claim 1, * indicates a stereocenter, and the proportion of the represented epimer in relation to the corresponding other epimer is greater than 50%.

13. The method according to claim 12, wherein the ketone of the general formula (X) or (XI) has the structure of the formula (XII) or (XIII) and R¹ to R⁵ are as defined in claim 1.

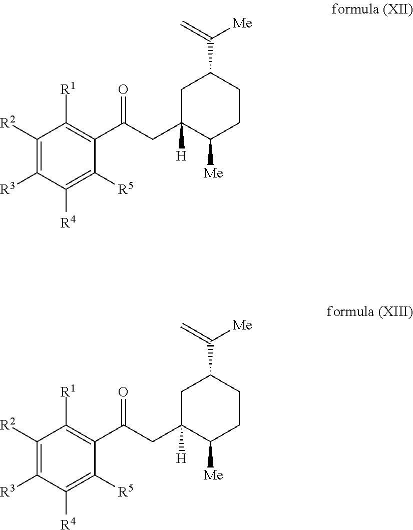

14. A ketone of one of the general formulae (X) or (XI), 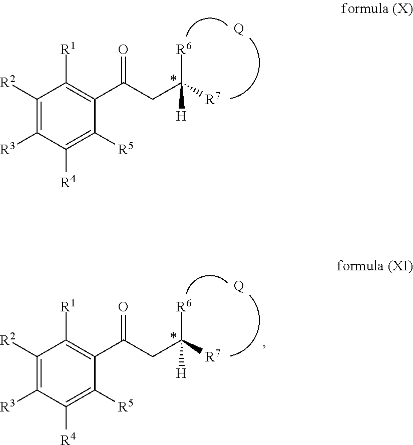
wherein R¹ to R⁵, in each case independently of one another, stand for hydrogen, halogen, NO₂—, —OH, —NR′R″R′″, or for linear, branched, or cyclic, saturated or unsaturated radicals with up to 15 carbon atoms, wherein R′, R″, and R′″, in each case independently of one another, are selected from hydrogen and up to 15 carbon atoms, wherein the carbon atoms are linear, branched, cyclic, saturated, unsaturated alkyl radicals, or combinations thereof, and wherein the carbon atoms of the ring of formula (I) linked to R¹ and R² or R² and R³ can be a common component of an annealed five-, six- or seven-membered cycloalkyl, aryl, heteroaliphatic or heteroaryl ring, wherein the radicals R¹ and R² or R² and R³, in each case independently of one another, are completely or partly an integral component of the annealed ring; and wherein one or more hydrogen atoms, methyl groups, methylene groups, methine groups, quaternary carbon atoms, or combinations thereof of the radicals R¹ to R⁵ can, in each case independently of one another, be substituted by heteroatoms; and
wherein R⁶ to R⁷, in each case independently of one another, stand for a secondary, tertiary, or quaternary carbon atom; wherein Q stands for an R⁶ to R⁷-bridging substituted or unsubstituted group with up to 11 carbon atoms, and * indicates a stereocenter, and the proportion of the represented epimer in relation to the corresponding other epimer is greater than 50%.

15. An agent comprising a ketone of the general formula (X) or (XI), 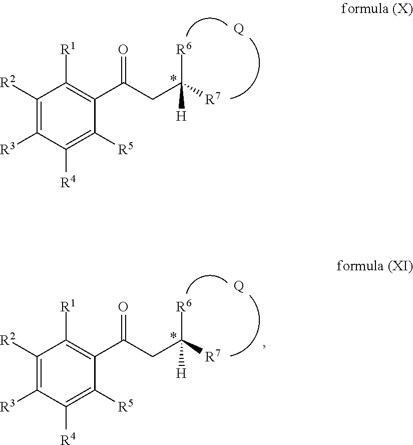
wherein R¹ to R⁵, in each case independently of one another, stand for hydrogen, halogen, NO₂—, —OH, —NR′R″R′″, or for linear, branched, or cyclic, saturated or unsaturated radicals with up to 15 carbon atoms, wherein R′, R″, and R′″, in each case independently of one another, are selected from hydrogen and up to 15 carbon atoms, wherein the carbon atoms are linear, branched, cyclic, saturated, unsaturated alkyl radicals, or combinations thereof, and wherein the carbon atoms of the ring of formula (I) linked to R¹ and R² or R² and R³ can be a common component of an annealed five-, six- or seven-membered cycloalkyl, aryl, heteroaliphatic or heteroaryl ring, wherein the radicals Wand R² or R² and R³, in each case independently of one another, are completely or partly an integral component of the annealed ring; and wherein one or more hydrogen atoms, methyl groups, methylene groups, methine groups, quaternary carbon atoms, or combinations thereof of the radicals R¹ to R⁵ can, in each case independently of one another, be substituted by heteroatoms; and
wherein R⁶ to R⁷, in each case independently of one another, stand for a secondary, tertiary, or quaternary carbon atom; wherein Q stands for an R⁶ to R⁷-bridging substituted or unsubstituted group with up to 11 carbon atoms, and * indicates a stereocenter, and the proportion of the represented epimer in relation to the corresponding other epimer is greater than 50%.

16. The agent of claim 15, wherein the agent is selected from the group consisting of
a washing agent, a cleaning agent, a cosmetic agent, an adhesive, a printing ink, or combinations thereof.

17. A method of prolonging the scent impression on a surface treated with the ketone of claim 1, wherein the method comprises:
treating the surface with the ketone and a washing agent a cleaning agent, a cosmetic agent, an adhesive, a printing ink, or combinations thereof.
